(19) United States
(12) Patent Application Publication
(10) Pub. No.: US 2005/0288696 A1
(43) Pub. Date: Dec. 29, 2005

(54) Device for separating the epithelial layer from the surface of the cornea of an eye

(76) Inventors: Ioannis G. Pallikaris, Kalessa (GR); Harilaos Ginis, Paster (GR); Scott M. Hampton, Cumming, GA (US); Stephen P. Woods, Hitchin (GB)

Correspondence Address: BRINKS HOFER GILSON & LIONE, P.O. BOX 10395, CHICAGO, IL, 60610, US

(21) Appl. No.: 10/971,727

(22) Filed: Oct. 22, 2004

Related U.S. Application Data
(63) Continuation-in-part of application No. 10/098,167, filed on Mar. 12, 2002.
Application No. 10/098,167 is a continuation-in-part of application No. 09/911,356, filed on Jul. 23, 2001.

(51) Int. Cl. A61F 9/00

(52) U.S. Cl. 606/166

(57) ABSTRACT

A separator that includes a body and a leading edge portion. The leading edge portion includes an upper planar surface oriented at an angle ranging from approximately 40° to 90° relative to a horizontal plane and a lower planar surface oriented at an angle ranging from approximately 0° to 30° relative to the horizontal plane, wherein the upper planar surface and the lower planar surface intersect one another so as to define a blunt leading edge and the leading edge portion is made of a material that has a hardness so as to separate an epithelial layer of an eye from an cornea stroma of said eye without substantially damaging said epithelial layer and wherein said blunt leading edge is incapable of cutting said stroma.

### REFERENCE TO EARLIER FILED APPLICATIONS

Applicants claim, under 35 U.S.C. § 119(e), the benefit of priority of the filing date of Aug. 6, 2004, of a U.S. Provisional Patent Application filed on the aforementioned date having the title “Separator for Corneal Epithelium” listing Scott M. Hampton, Stephen Woods and Harilaos Ginis as inventors and having an attorney docket no. of 2N04.1-040, the entire contents of which are incorporated herein by reference and in addition the present application is a continuation-in-part application of U.S. patent application Ser. No. 10/098,167, filed on Mar. 12, 2002, which is a continuation-in-part application of U.S. patent application Ser. No. 09/911,356, filed Jul. 23, 2001, the entire contents of each of which are incorporated by reference herein.

### BACKGROUND

LASIK (Laser-Assisted In Situ Keratomileusis) is a surgical procedure intended to reduce a person's dependency on glasses or contact lenses. LASIK permanently changes the shape of the cornea, the clear covering of the front of the eye, using an excimer laser. A device, called a microkeratome, is used to cut a flap in the cornea. A hinge is left at one end of this flap. The flap is folded back revealing the stroma, the middle section of the cornea. Pulses from a computer-controlled laser vaporize a portion of the stroma and the flap is replaced. It is important that the knife used during the LASIK procedure is sharp, otherwise the quality of the procedure and the healing time are poor. Additionally the knife has to be sharp in order to produce consistent and reproducible flaps. FIG. 20 is a diagram showing a perspective view of a known blade 2000 that can be used for the cutting involved in the LASIK procedure.

There are some complications related to the use of microkeratomes. Common complications include the creation of an irregular flap, for example, a half flap, a buttonhole, or a total cup. These complications represent irregular incisions of the cornea, a situation that can permanently degrade visual performance.

Alternatively, PRK (Photo-Refractive Keratectomy) which is a technique developed earlier than LASIK may be used to correct the curvature of the cornea. In PRK a physician scrapes away the superficial layer, e.g., the epithelium, of the cornea. After the superficial layer is removed, laser treatment is applied on to the exposed surface of the cornea. A drawback of PRK, however, is that the healing period for the eye typically lasts for a week, much longer than the healing period of LASIK. Also, the patient experiences some pain during healing. Typically in PRK a disposable contact lens is used to cover the treated area of the cornea and help reduce postoperative pain.

In another technique, LASEK (Laser Epithelial Keratomileusis) the epithelial layer is separated from the surface of the cornea in a manner that the separated epithelial layer can be preserved. First, the epithelium is treated with and alcohol solution to partially devitalize it. Once the exact surface area of treatment is determined, a few drops of a weak alcohol solution is applied to the surface of the cornea and allowed to stay in contact with the epithelium for a few seconds. This weak alcohol solution is then rinsed off the surface of the eye. The function of the weak alcohol solution is to loosen the epithelial layer (50 microns) and to allow it to be peeled back by a handheld spatula in a sheet of epithelial cells, thereby exposing the underlying cornea. This is not to be confused with LASIK, which actually uses a microkeratome instrument to create a flap of both epithelium and the front part of the stromal tissue measuring anywhere between 130 to 180 microns.

In LASEK, the epithelium-only layer is laid back in a similar fashion to LASIK, but consists of only epithelium, not corneal stroma. Once the epithelial cells have been laid out of the way, the laser is applied to the surface of the cornea in the exact same fashion as in PRK. Once the laser treatment has been completed, the epithelial layer is laid back into place and a soft contact lens is placed over the eye as in PRK. The epithelial cells, which were partly devitalized by the weak alcohol solution, are laid over the treatment area and may serve as a facilitator of new epithelium healing underneath. The alcohol-devitalized epithelium falls off the eye, similar to a scab, in 5-10 days. These devitalized epithelial cells do not become the new surface of the eye, but simply serve as a protective agent in addition to the contact lens to facilitate comfort and healing of the new underlying epithelium. Alcohol treatment of the epithelium results in a severe amount of epithelial cell loss, a fact that may render the epithelial disk not usable, due to the reduced durability and adhesion on to the cornea.

Thus, there is a need for an automated corneal epithelium separator that addresses the above problems by separating the epithelial layer as a whole in a mechanical way, not chemical.

### BRIEF SUMMARY

To help correct an imperfect vision of a patient's eye, an automated mechanical device separates the epithelial layer from the cornea of a patient's eye from the cornea. After the epithelial layer is separated from the cornea, a laser is used to help correct imperfections in the cornea. Thereafter, the epithelial layer is placed back on the cornea to reduce the visual rehabilitation period and reduce postoperative pain.

A first aspect of the present invention regards a separator that includes a body and a leading edge portion. The leading edge portion includes an upper planar surface oriented at an angle ranging from approximately 40° to 90° relative to a horizontal plane and a lower planar surface oriented at an angle ranging from approximately 0° to 30° relative to the horizontal plane, wherein the upper planar surface and the lower planar surface intersect one another so as to define a blunt leading edge and the leading edge portion is made of a material that has a hardness so as to separate an epithelial layer of an eye from an cornea stroma of said eye without substantially damaging said epithelial layer and wherein said blunt leading edge is incapable of cutting said stroma.

A second aspect of the present invention regards a method of processing an eye of a patient for a corrective procedure by moving a separator relative to a cornea of an eye and separating an epithelial layer associated with the cornea. The separated epithelial layer defines a hinge on the cornea so that a free end of the epithelial layer is pivoted about the hinge so as to be positioned near an eyebrow of the patient.

A third aspect of the present invention regards a method of processing an eye of a patient for a corrective procedure by moving a separator along a linear direction relative to a cornea of an eye and automatically and electronically controlling the distance traveled by the separator along the linear direction.

Each of the above aspects provides the advantage of reducing the visual rehabilitation period and postoperative pain associated with epithelial layer replacement procedures.

### BRIEF DESCRIPTION OF THE DRAWINGS

FIG. 1A is a diagram showing a side view of an eye and an epithelial separator device with a separator located in a first position according to the preferred embodiments.

FIG. 1B is a side view of an embodiment of the separator used with the epithelial separator device of FIG. 1A.

FIG. 2 is a diagram showing a top view of the eye and the separator located in a first position according to the preferred embodiments.

FIG. 3 is a diagram showing a side view of the eye and the separator located in a second position according to the preferred embodiments.

FIG. 4 is a diagram showing a top view of the eye and the separator located in a second position according to the preferred embodiments.

FIG. 5 is a diagram showing a side view of the eye and the separator located in a third position according to the preferred embodiments.

FIG. 6 is a diagram showing a top view of the eye and the separator located in a third position according to the preferred embodiments.

FIG. 7 is a diagram showing a side view of the eye and the separator located in a fourth position according to the preferred embodiments.

FIG. 8 is a diagram showing a top view of the eye and the separator located in a fourth position according to the preferred embodiments.

FIG. 9 is a diagram showing a top view of the eye and the separator located in a fifth position according to the preferred embodiments, the separator is retracted after epithelial separation.

FIG. 10 is a diagram showing a top view of the eye with the separator removed.

FIG. 11 is a diagram showing a top view of the eye after ablations is performed with a laser.

FIG. 12 is a diagram showing a top view of the eye with the epithelium replaced on the eye.

FIG. 13 is a diagram showing a top view of the eye with the epithelium smoothly stretched into place.

FIG. 14 is a diagram showing a side view of the eye and the epithelial separator device including a rotating drum.

FIG. 15 is a diagram showing a front view of the eye and the epithelial separator device including the rotating drum.

FIG. 16 is a diagram showing a top view of the eye and the epithelial separator device including the rotating drum.

FIG. 17 is a diagram showing a drum according to one embodiment.

FIG. 18 is a diagram showing a drum according to another embodiment.

FIG. 19 is a diagram representing a side view of a separator removing the epithelial layer from the Basal membrane of the eye.

FIG. 20 is a diagram showing a perspective view of a known blade.

FIG. 21 is a diagram showing a side view of a separator's leading edge according to an embodiment.

FIG. 22 is a diagram showing a side view of a separator's leading edge according to another embodiment.

FIG. 23 is a diagram showing a side view of a separator's leading edge according to yet another embodiment.

FIG. 24A shows a top perspective view of a second embodiment of an epithelial separator according to the present invention.

FIG. 24B shows a bottom perspective view of the epithelial separator of FIG. 24A.

FIG. 25 is a side view of an embodiment of the separator used with the epithelial separator of FIGS. 24A-B.

FIG. 26 shows a perspective view of an embodiment of a guard to be used with the epithelial separators of FIGS. 1A-B and 24A-B.

FIG. 27 shows a top perspective view of the epithelial separator of FIG. 24 when employing an embodiment of an ablator at a first position.

FIG. 28 shows a top perspective view of the epithelial separator of FIG. 24 when employing the ablator of FIG. 30 at a second position.

FIG. 29 shows a bottom perspective view of the epithelial separator of FIG. 24 when employing the ablator of FIG. 27.

FIG. 30 is a diagram showing a side view of a separator's leading edge according to another embodiment.

FIG. 31A is a diagram showing a side view of a separator according to yet another embodiment.

FIG. 31B is an enlarged side view of a portion of a leading edge of the separator shown in FIG. 31A.

FIG. 31C is an enlarged side view of a portion of a leading edge of another embodiment of a separator.

FIG. 32 is a diagram showing a perspective view of a wire that could be used as a separator according to a preferred embodiment.

FIG. 33 shows a perspective view of an exemplary machine that is used to condition a separator according to one embodiment.

FIG. 34A shows a front view of the machine of FIG. 33 including the separator.

FIG. 34B schematically shows a side view of the machine of FIG. 33 including the separator.

FIG. 35 shows a side view of one embodiment of a device for separating and preserving an epithelial layer.

FIG. 36 shows a top view of the device of FIG. 35.

### DETAILED DESCRIPTION

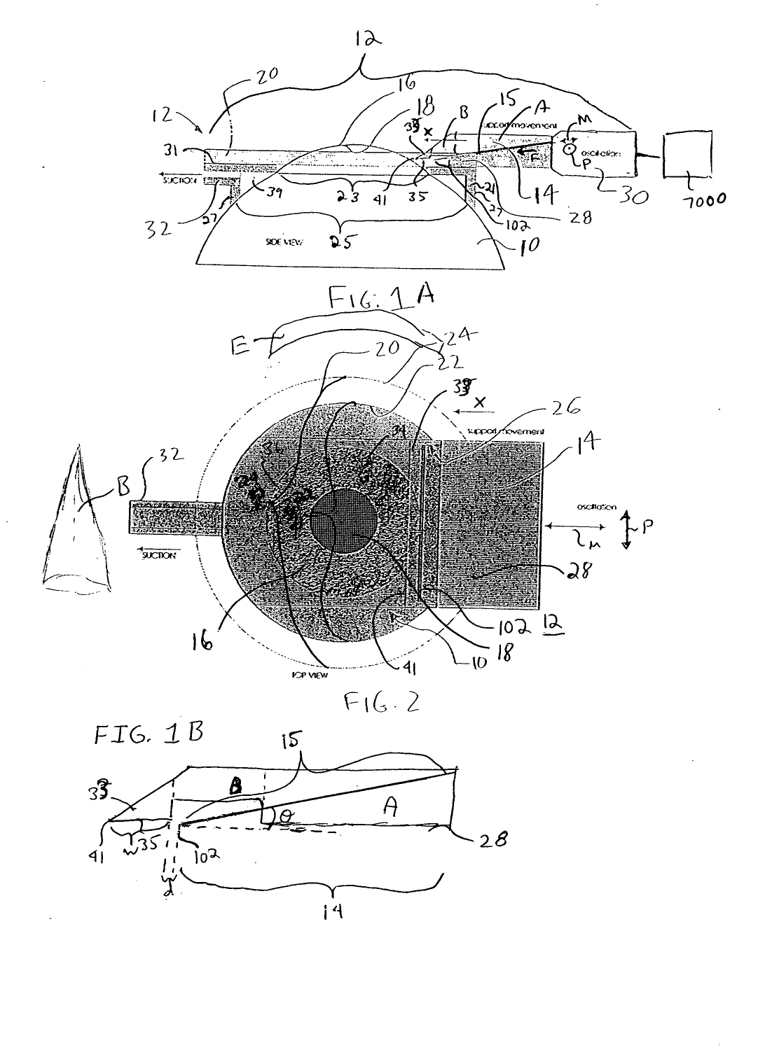

To help correct an imperfect vision of a patient's eye, an epithelial separator device 12 (as shown in FIGS. 1-9) separates at least a portion of the surface area of the epithelial layer 16 positioned upon the corneal surface 18. The portion of the surface area removed corresponds to the area of the eye to be ablated by the laser. The separation preferably involves the full thickness of the layer 16 ranging from the portion adjacent to the corneal surface 18 to the exterior surface of the layer 16. See FIG. 19)

Note that the corneal surface 18 from which the epithelial layer 16 is separated according to the present invention is deemed to be composed of stroma material that is defined to include Bowman's layer and corneal stroma either separately or in combination with one another. In other words, the various separation processes described herein with respect to the present invention can be performed either when Bowman's layer is positioned upon the corneal stroma or when the Bowman's layer is absent from the eye in question.

The separation process described above and below with respect to the present invention provides several advantages over LASIK and LASEK. In particular, the device 12 separates the epithelium without substantial epithelial cell loss. That is, epithelial cell loss is often less than 5-10% loss, to ensure viability; which compares favorably to prior techniques that employ a sharp blade that removes the layer 16 along with a substantial amount of corneal stroma material attached thereto by cutting into the cornea underlying below layer 16. The low epithelial cell loss achieved by device 12 therefore achieves a more viable epithelial layer 16 after replacement on the corneal surface 18, when compared with the previously described LASEK alcohol-assisted separation. Such a more viable epithelial layer 16 provides improved healing for the eye when compared with LASEK after the laser ablation is performed and the epithelial layer 16 is placed back on the corneal stroma or Bowman's layer.

Another advantage of the present invention is that no stroma material is removed from the corneal surface 8 when compared with LASIK that involves having a sharp blade cutting into and removing stroma material. The depth of the LASIK cut is determined by the spacing of the applanator that exists in all LASIK mikrokeratomes. The failure of the present invention to cut into and remove stroma material results in the stroma material being more mechanically sound to endure the process than the stroma material remaining in a LASIK procedure.

Another advantage of the present invention is that there is less risk in creating optical irregularities in separation of the epithelial layer 16 in accordance with the present invention when compared with the formation of the previously mentioned irregular flap formed in LASIK. This is due to the epitheliium is something that naturally is renewed every few days. If the epithelial layer 16 is removed, it will grow back in place. If something goes wrong with separating the epithelial flap there is no real problem; the surgeon can throw it away and it will grow again. But if with the help of the present invention, the epithelial layer 16 can be harvested as a whole sheet, it can be repositioned so as to cover/protect the ablated site. In a few days, the epithelial layer 16 placed on the ablated site is replaced by new epithelial cells that have grown in its place. The separated cells have died and fallen off as they would naturally do. In contrast, in LASIK, the microkeratome takes a piece of corneal stroma, which is more “permanent” tissue and replaced on the eye after ablation If something goes wrong with the “permanent” tissue portion of the flap created, the patient will have to live with this irregularity forever or undergo a number of corrective procedures including corneal transplantation.

Other advantages of the present invention include the simplicity of the procedure, the speed of the procedure and the fact that it requires much less surgeon training than other techniques.

Moreover, some embodiments of the separator devices 12′, 2700, such as shown in FIGS. 14-18 and FIGS. 35-36, include a holder, such as a drum 42 or film 2740, holds and in some embodiments preserves—with a hydrating and/or a conditioning substance—a portion of or all of the separated epithelial layer 16, such as in the form of a disk 34, without rupturing the layer 16.

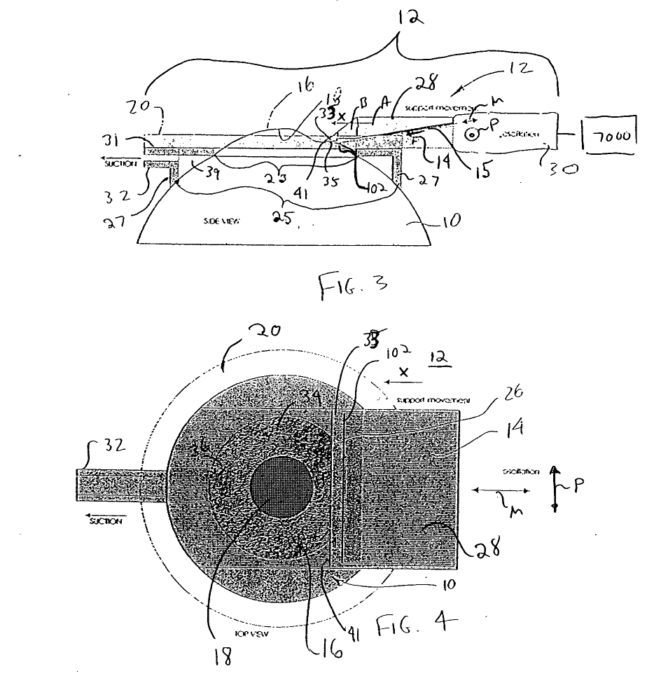

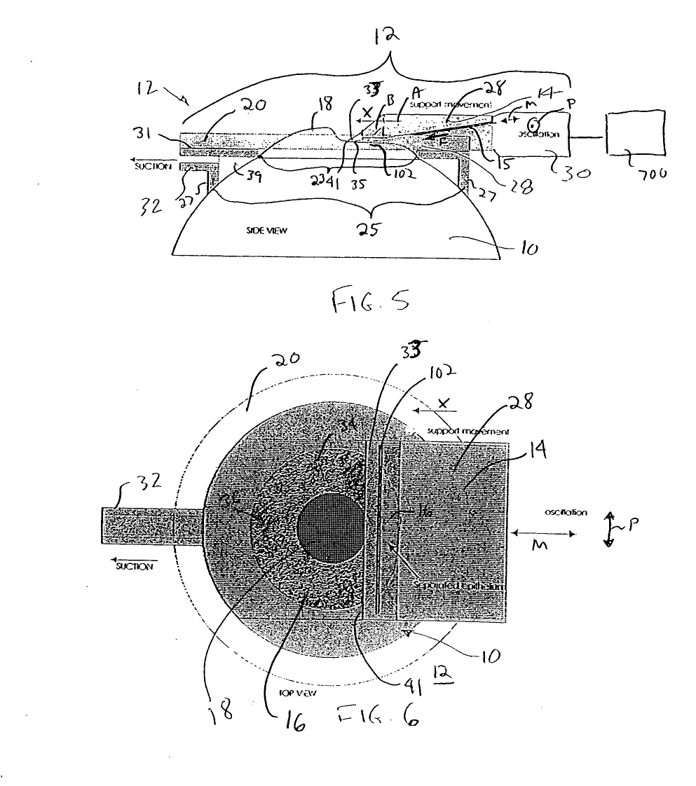

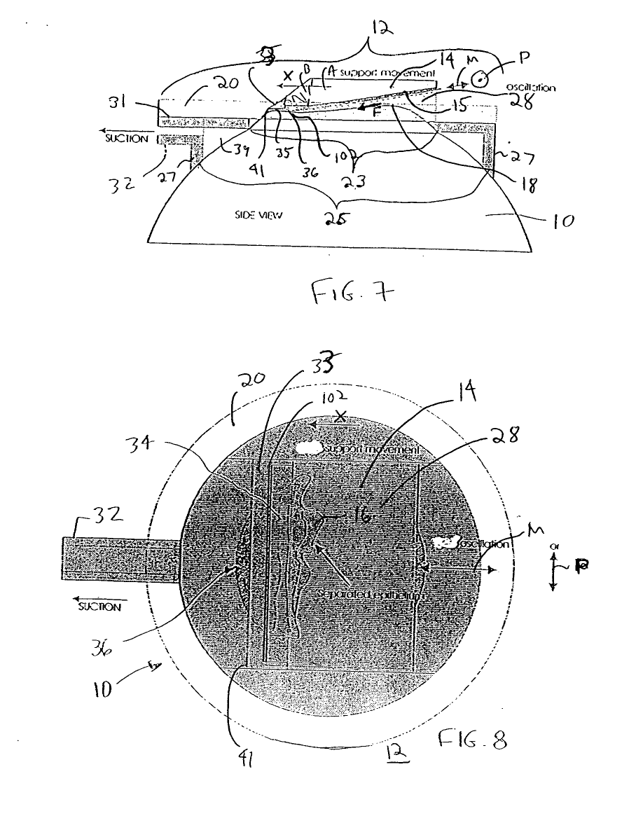

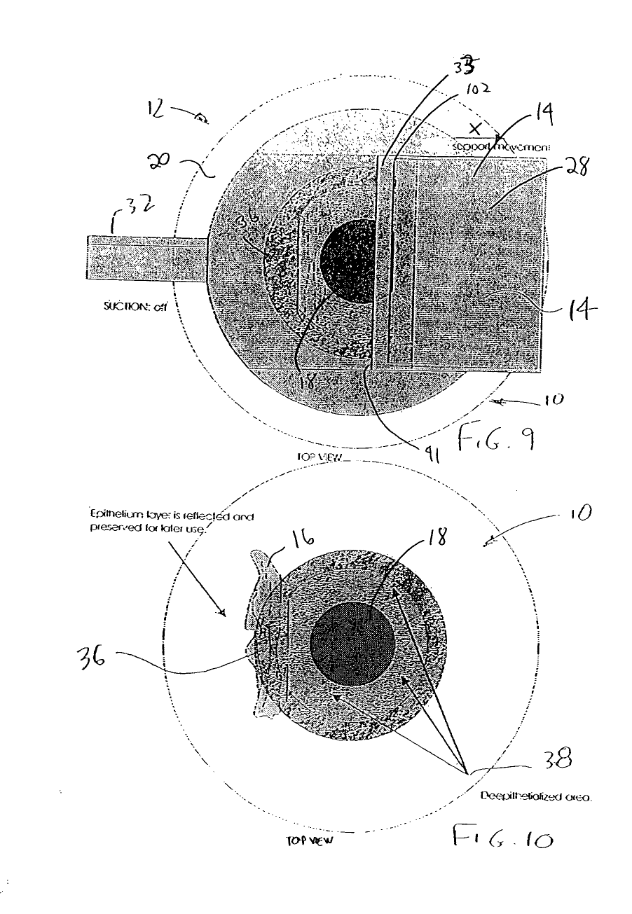

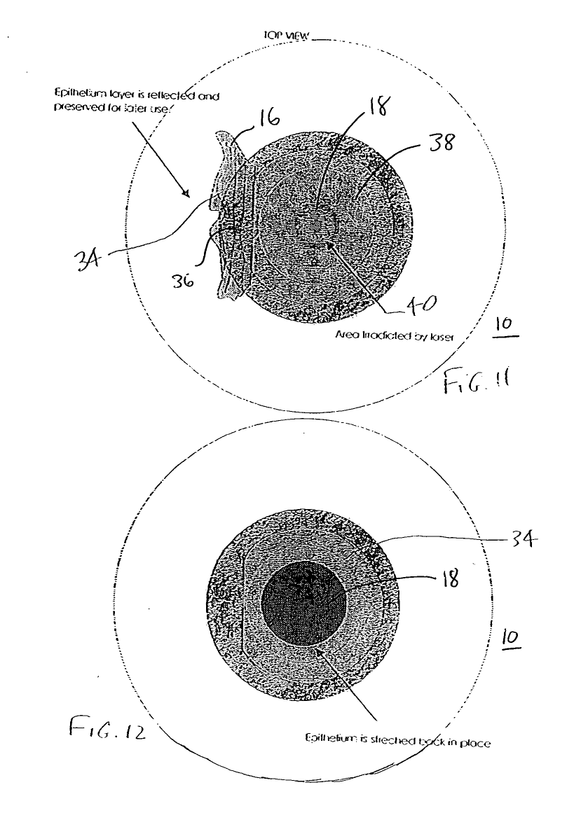

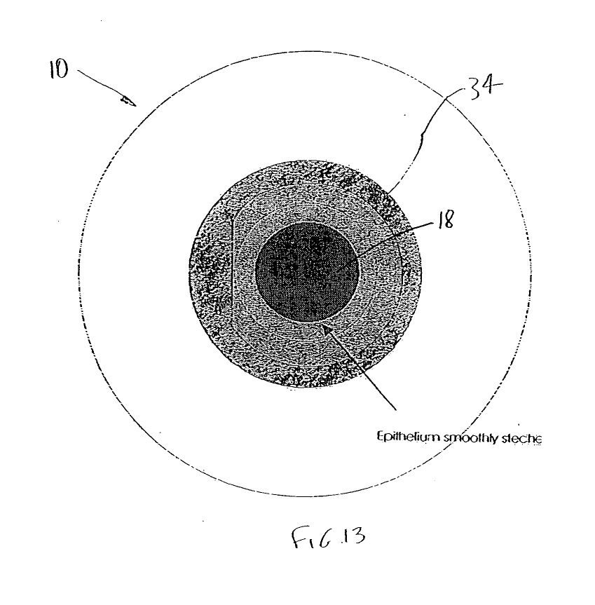

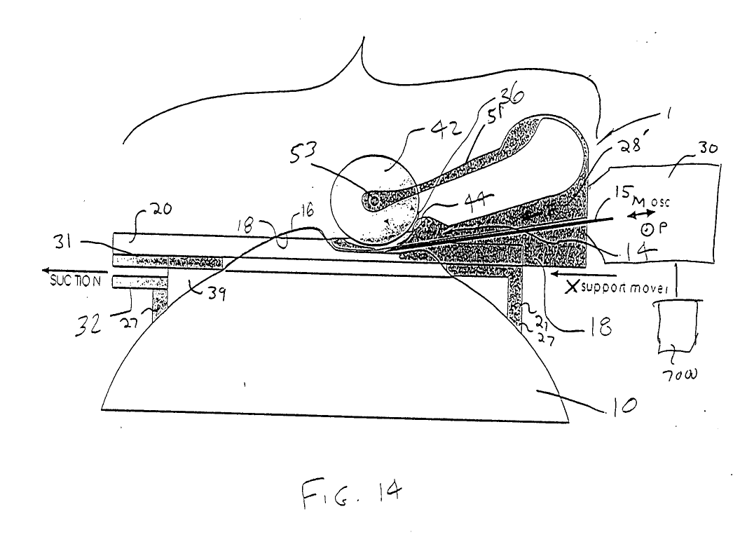

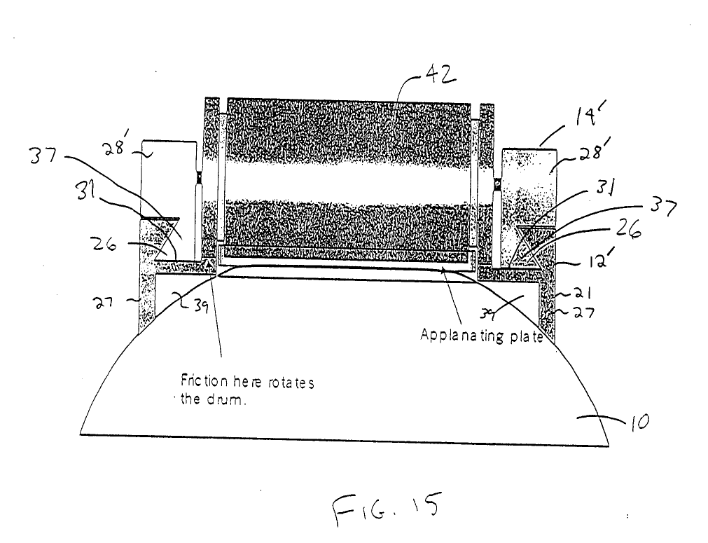

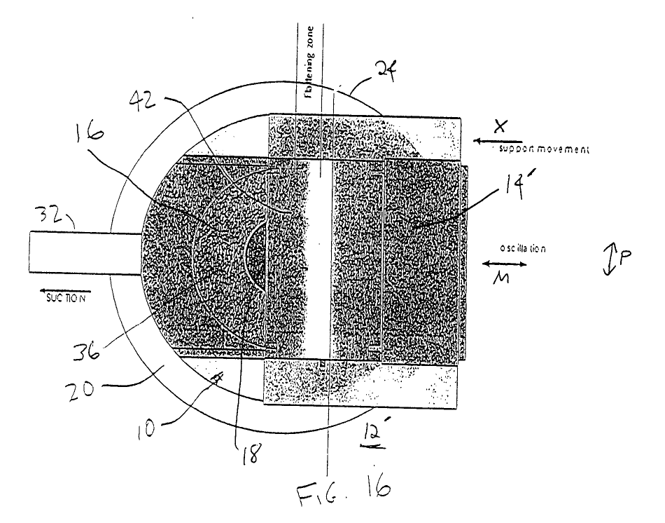

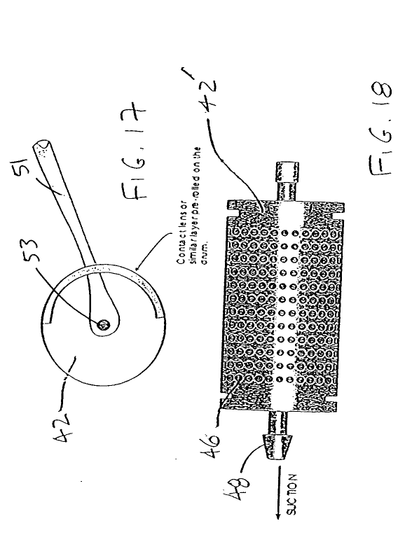

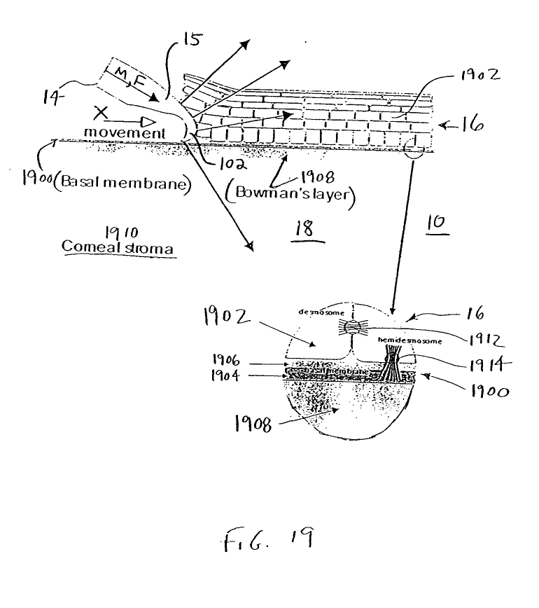

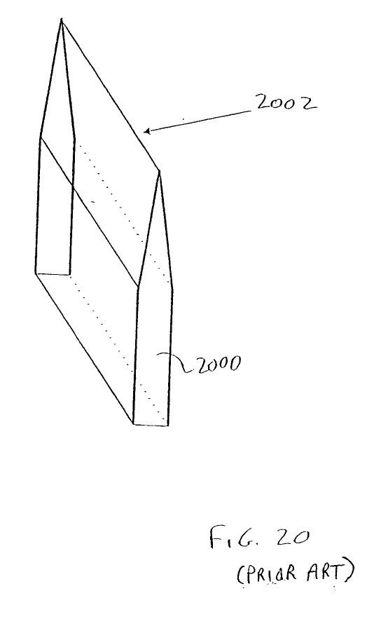

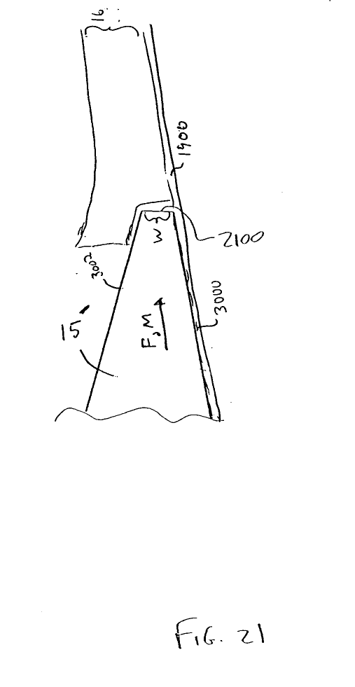

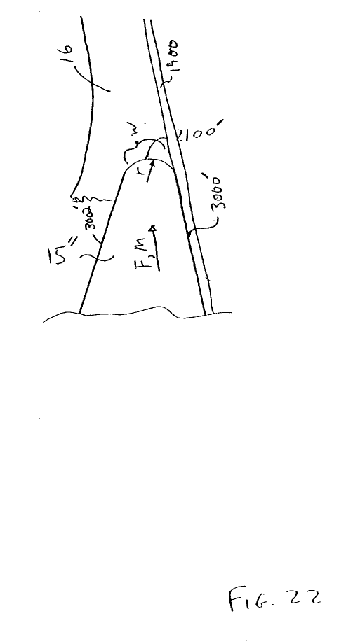

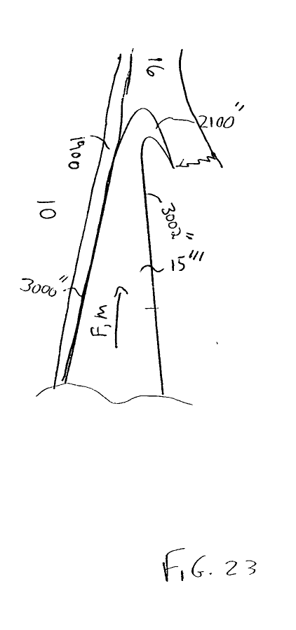

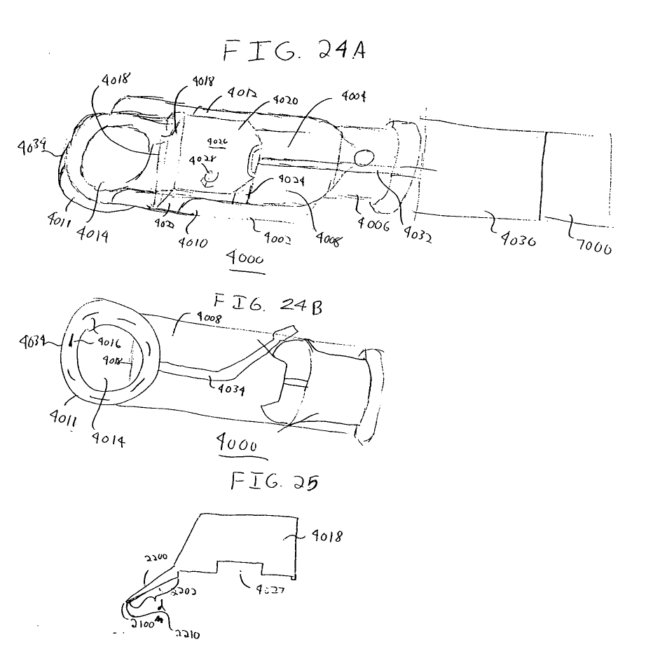

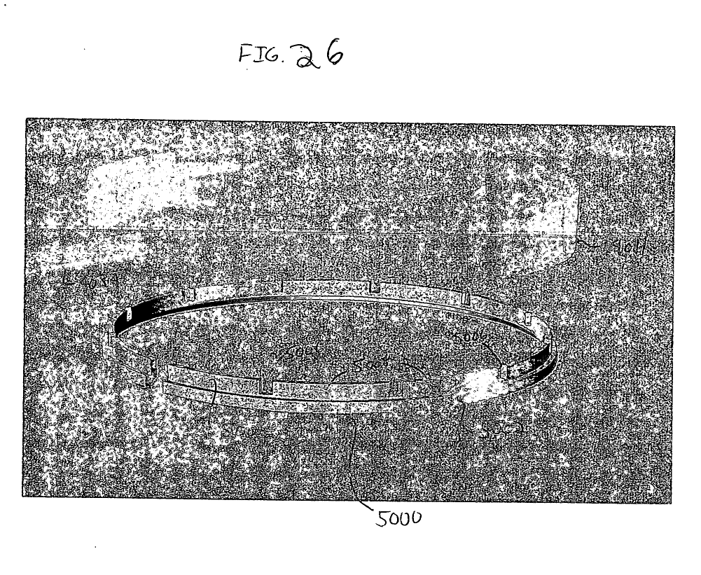

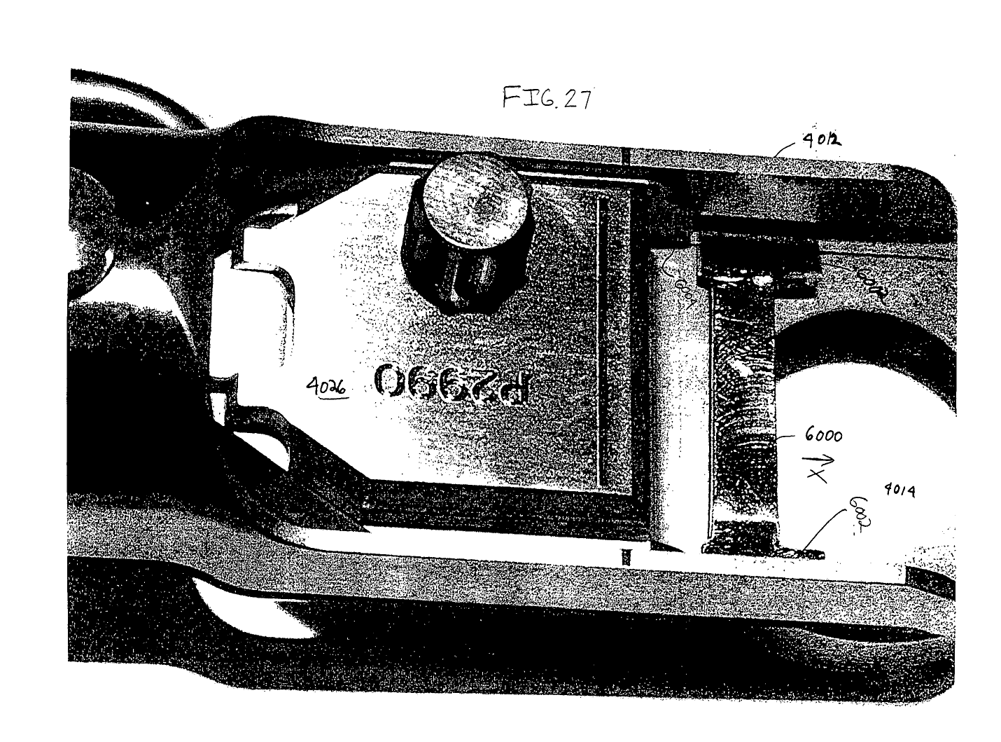

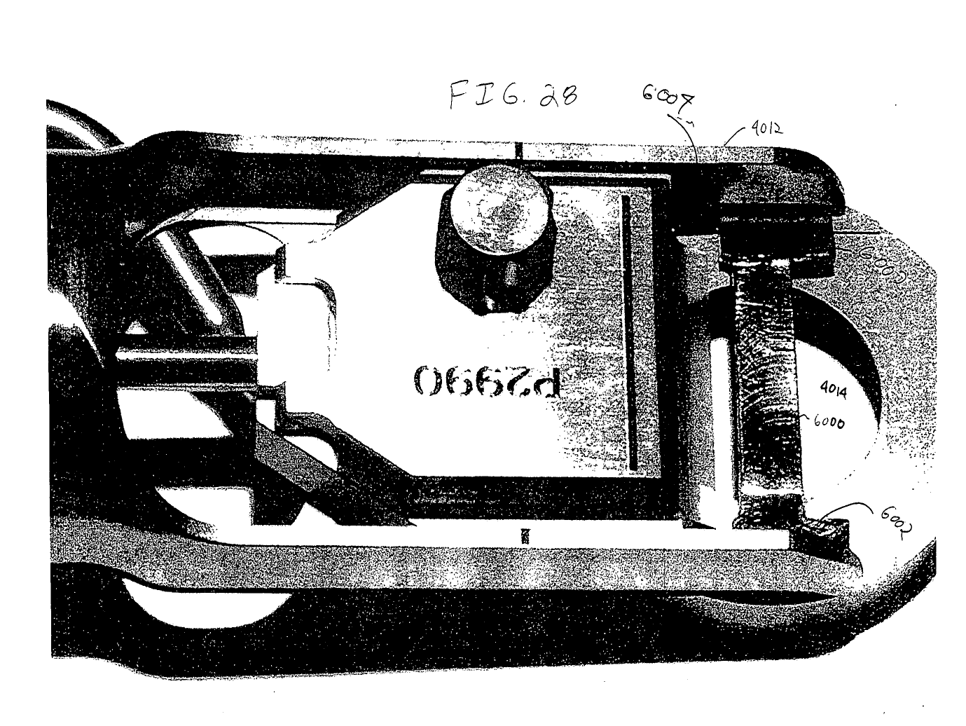

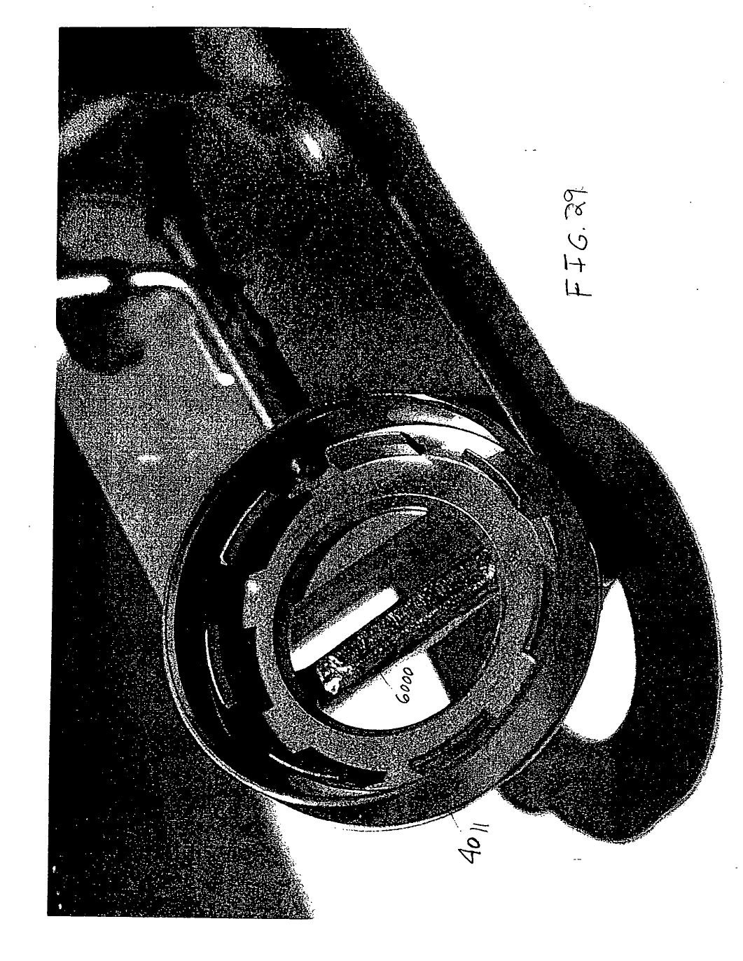

FIG. 1A shows an eye 10 of a patient and an epithelial separator device 12. The epithelial separator device 12 includes a separator 14, such as a separator support 28 that supports a blunt edge 102, a wire or a knife. The separator 14 is structurally designed such that when initially contacting a contact edge of a tissue layer, such as the epithelial layer 16, and when appropriate forces and/or appropriate oscillations, such as 6000 Hz to 15,000 Hz, are applied to the separator, the separator 14 is able to push one or more layers of tissue, such as the layer 16, off of the corneal surface 18 while simultaneously not penetrating/cutting into the corneal surface 18. Note that the blunt edge 102 initially oscillates side-to-side with an amplitude ranging from 1.25 mm to 1.6 mm along direction P of FIGS. 1A and 2 and is translated along direction X so that it punctures through the epithelial layer 16 until it contacts the corneal surface 18. At this point, the blunt edge 102 continues its side-to-side oscillations and translational movement along direction X so that the blunt edge 102 pushes the layer 16 in the manner described above. Note that the separation of the epithelial layer 16 from the corneal surface 18 is the result of the blunt edge 102 and the separator 14 creating a cleavage plane between two materials with different mechanical properties, namely the epithelial layer 16 and the corneal surface 18.

At this point it would be helpful to understand a few concepts as to how the blunt edge 102 interacts with the epithelial layer 16. First, as defined throughout this application, the act of cutting will be defined as the act of separating a physical object into two portions, through the application of an acutely directed force onto a cutting surface of a cutting implement so as generate compressive and shearing forces onto the physical object that cause the physical object to separate along a path defined by the cutting surface. This is in contrast to the previously mentioned puncturing by the blunt edge 102, wherein such puncturing does not cause the layer 16 to separate along a path defined by blunt edge 102.

It is well known that cutting as defined above and throughout this description occurs when the cutting area has a hardness greater than the physical object and the total stress generated through the cutting surface exceeds the ultimate strength of the physical object being cut. Since the total stress, σ, is defined to be σ=F/A, wherein F equals the force applied to the cutting area and A equals the size of the cutting area, the stress of a cutting tool can be manipulated by changing either the amount of force applied and/or the size of the cutting area.

During the previously mentioned pushing process, the separator 14, such as via blunt edge 102 of separator support 28, pushes the contact edge of the tissue towards a second edge of the tissue positioned opposite to the contact edge. As shown in FIG. 19, the blunt edge 102 forces the contact edge to be lifted up, separate from corneal surface 18 and travel along a portion of the top of the plate 15 as the separator translates along direction X. Note that the previously mentioned top portion of the plate 15 acts as a support surface for the separated epithelial layer 16. The pushing process is such that the blunt edge 102 is incapable of cutting into the corneal surface 18 and the tissue layer is substantially preserved after the pushing process is complete. In some cases, the amount of damaged epithelial cells during the process of the present invention may be less than that caused by epithelial cells that undergo a LASEK procedure.

At the end of the pushing process, the separator 14 has traveled either the entire diameter of the corneal surface 18 so as to remove the entire tissue layer or has traveled a substantial portion of the diameter so that the tissue layer at the second edge is still adhering to the corneal surface 18.

In the case of the separator 14 shown in FIG. 1B, the separator includes a separator support 28 in the form of a rectangular block A. Note that the separator support 28 may be made of a number of rigid and sterile materials, such as metals and plastics. In the case of plastics, the material can be transparent so that the user can see the portion of the eye 10 directly below the separator support 28.

The rectangular block A is made of a material that has sufficient hardness such that it does not deform during the separation process. An example of an acceptable material would be PMMA (Polymethylmethacrylate), ceramic or a well-known surgical instrument metal. The rectangular block A has a height of approximately 3 mm, a length of approximately 12 mm and a depth of approximately 6 mm. Note that the bottom of rectangular block A is polished and/or coated with a material, such as paryleme, in order to reduce friction between the bottom and the eye.

As shown in FIG. 1B, the separator 14 further includes a planar-like plate 15 that is positioned within an angled recess formed in the block A of the separator support 28. The recess is preferably dimensioned to have a height/thickness and width that substantially corresponds to the height/thickness and width of the plate 15 so that the plate 15 is constrained to move along direction M shown in FIG. 1A. If movement of the plate 15 in a direction P perpendicular to direction M is desired, then the width of the recess can be enlarged by a desired amount.

Note that the plate 15 may be made of a number of rigid and sterile materials, such as polymers, metals and plastics. The plate 15 is designed to be as light as possible while having the properties of avoiding vibration and being able to push the epithelium layer 16 during the separation process. An example of a suitable material is PMMA, wherein in such a case the plate would have a mass of approximately 0.5 g. Note that the plate 15 can be polished and/or coated with a friction reduction material, such as paryleme, in order to reduce friction between the plate 15 and the eye

The plate 15 has a width ranging from approximately 250 μm to approximately 350 μm and has a thickness that can range from the thickness of a single cell layer of the epithelial layer 16 to the total thickness of layer 16. More preferably, the plate 15 has a thickness between two to three cell layers in thickness. The plate 15 extends at an angle θ that ranges from 0° to 30° At a distal end of the plate 15, a blunt edge 102 is shaped and designed so that upon being subjected to certain forces and oscillations explained below it will contact one or more layers of the tissue to be removed, such as the epithelial layer 16, without penetrating into corneal tissue located below the layers of tissue. In other words, during use of the device 12, the edge 102 is not sharp enough to penetrate into corneal tissue so as to cut or excise such tissue during operation of the epithelial separator device 12. Thus, the blunt edge 102 is in direct contrast to the leading edge 2002 of the blade 2000 described previously with respect to FIG. 20 in that edge 2002 is sharp and would risk cutting the corneal surface 18 if used to separate layer 16 from the corneal surface 18. Note that the blunt edge 102 is that portion of the distal end of the plate 15 that makes contact with the epithelial layer 16 after the initial puncturing through the layer 16 and during the subsequent separation process. The blunt edge 102 also includes adjacent portions of the plate 15 that are in contact with the corneal surface 18 in a non-cutting manner. Thus, the plate 15 defines an edge that is both blunt with respect to the epithelial layer 16 (because it does not substantially damage the layer 16 during the moving/separation process) and the corneal surface 18/cornea stroma (because the surface of the plate 15 contacting the cornea stroma does not damage the stroma during the separation process).

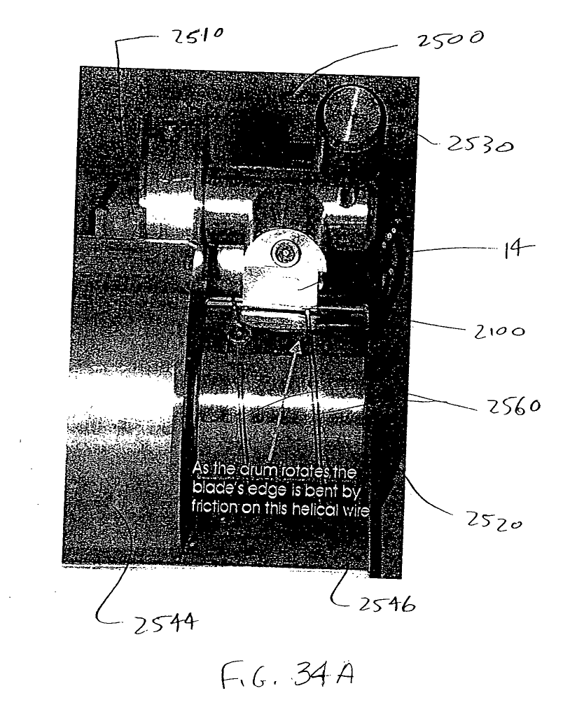

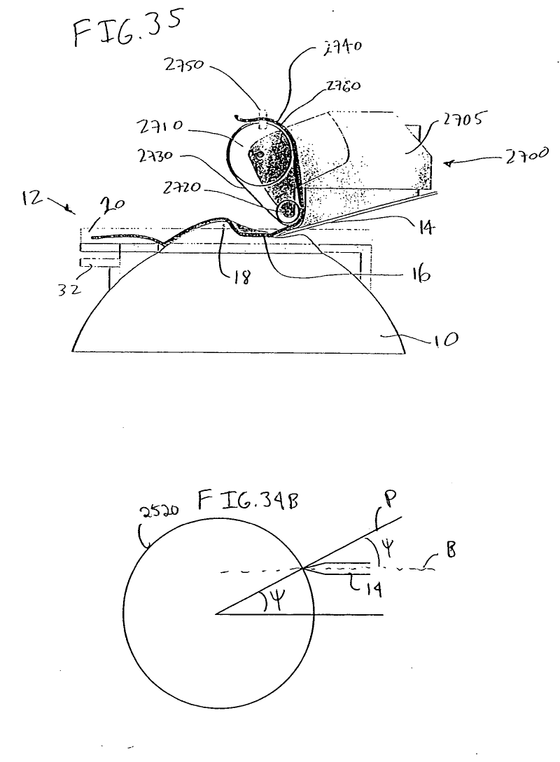

As mentioned previously, the ability for an edge to cut is related to the stress that it can create through its surface. Since the stress is inversely proportional to the surface area that applies the force, enlarging the surface area that is in contact with the corneal surface 18 would reduce the stress. Enlarging the surface area sufficiently will prevent the edge from cutting and so the edge would then qualify as being “blunt” as defined in the present application. For example, the prior art “sharp” edge 2002 shown in FIG. 20 can be converted to a “blunt” edge 102 by applying the edge 2002 to the machine 2500 shown in FIGS. 33-34 and described later on. In particular, the machine 2500 bends different portions of the initial edge at any one time so that a rounded blunt edge is formed. FIG. 23 gives an example of such bending and shows that the surface area of the rounded blunt edge 2100″ is greater than the area of the tip of the plate 15′″.

Another possibility is to lower the force applied to the corneal surface 18. Assuming that the cutting force is the perpendicular component of the force, F, transmitted by the plate 15 that is oriented at an angle θ, the cutting force has a magnitude of Fsin θ. Thus, making the angle θ as small as possible, such as zero degrees, would reduce the cutting force. In this situation, the plate 15 could have a rectangular side cross-section wherein a planar base of the plate 15 lies flat on the corneal surface (θ=0°) and the distal end's perpendicular side pushes the layer 16. While the right angled lower corner defined as the intersection of the base and the side is “sharp” it is not angled to apply any cutting force to the corneal surface 18. Thus, the right angled lower corner is effectively “blunt” with respect to its ability to cut the corneal surface 18. Using the same analysis, if plate 15 has a trapezoidal side cross-section and its base lies flat on the corneal surface, the angled corner between the base and side of the plate would also be considered “blunt” in cutting effectiveness while being “sharp” in appearance.

As shown in FIGS. 1A-B and 2, the separator support 28 of the separator 14 is integrally connected via a throat area B with a blunt distal end 33 that has a triangular-like cross-section. The bottom surface 35 of the distal end 33 is flat and is elevated and is offset with respect to the blunt edge 102 so that the edge 102 first contacts the outermost cells of the epithelial layer 16 (see FIG. 19) radially inwardly with respect to the circumferential edge of the layer 16. In particular, the blunt edge 102 is laterally offset from the rear edge of the bottom surface 35 a distance d that is approximately 300 μm and is positioned below the bottom surface 35 by a distance ranging from approximately 240 μm to 300 μm also. Note that the lateral offset is a function of how dull in appearance the edge is. For example, the duller the edge 102 is, the greater the lateral offset d is.

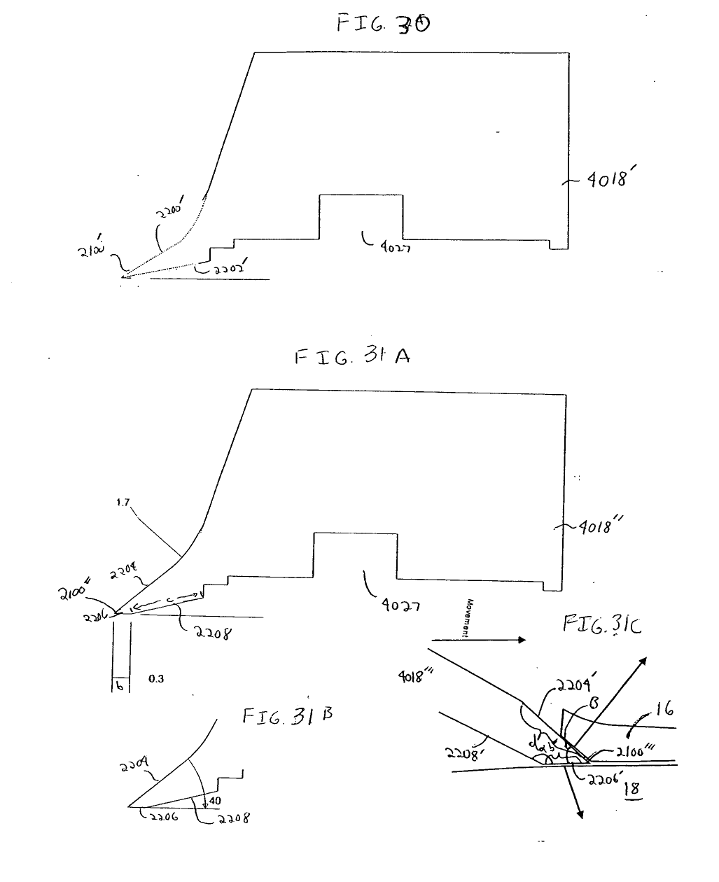

As shown in FIGS. 1A, 3, 5 and 7, the bottom surface 35 of the distal end 33 is positioned parallel to the bottom surface of rectangular block A and the top surface 31 of the housing 21. The rectangular bottom surface 35 has a width w of approximately 2 mm and a length as measured parallel to the front edge 41 of approximately 12 mm. As shown in FIG. 1A, the separator support 28 and the bottom surface 35 are initially positioned away from the eye of the patient. The separator support 28 and the bottom surface 35 are translated along direction X so that the bottom surface 35 makes initial contact with the exterior surface of the eye at a position inwardly of the edge of the epithelial layer 16 as shown in FIG. 3. During such contact, the distal end 33 and the bottom surface 35 compress the exterior surface of the corneal surface 18 and the epithelial layer 16. Such compression causes the layer 16 to be relatively flat from the blunt edge 102 to at least the front edge 41 of the distal end 33 as shown in FIGS. 3, 5 and 7. Thus, the bottom surface 35 of distal end 33 acts as an applanator.

Note that the bottom surface 35 of the distal end 33 needs to be polished as flat as possible so as to form a flat surface for the edge 102 and to decrease the possibility of decreasing the structural integrity of the layer 16 as the surface 35 translates along the top of the layer 16. The bottom surface 35 may be coated with a friction reduction material, such as paryleme, so as to reduce friction between the surface 35 and the eye. In addition, the edge 41 is made very blunt (more blunt than edge 102) by being very rounded in shape so as to avoid piercing the layer 16 during its movement.

Note that as an alternative embodiment, the distal end 33 can be removed and the separator support 28 can be altered so that the dull edge 102 is made less sharp in appearance and is rotated so that θ=0°. As mentioned previously, the plate 15 in this orientation can be used alone to perform both applanation of the corneal surface 18 and separation of layer 16 from the corneal surface 18.

As shown in FIG. 1, the separator device 12 includes an annular ring 20 made of a sterilizable and rigid material, such as a metal including titanium, The ring 20 is supported upon and attached to a housing 21 that defines an upper circular opening 23 and a lower circular opening 25. The upper opening 23 has a diameter of that corresponds to the inner diameter of the ring 20. The bottom opening 25 is defined by an outer wall 27, wherein the bottom opening has a diameter that ranges from 16 mm to 21 mm. The bottom of the outer wall 27 is curved so as to match the external radius of curvature of a portion of eye 10. In the alternative, the outer wall 27 can be slanted instead of being curved depending on the diameter of outer wall 27. The housing 21 is made of a sterilizable and rigid material, such as the material for ring 20, and has a height needed to fit onto the eye and so is based on the diameter of wall 27 and the average anatomic data for the eye. As shown in FIGS. 1A and 2, the bottom of the outer wall 27 is placed on the exterior surface of the eye 10 so that the top surface 31 of the housing 21 is parallel to a limbus of the eye 10. The bottom of outer wall 27 and top surface 31 may be slightly slanted to provide a better fit for certain individual eyes and to avoid having the bottom of outer wall 27 cut into the corneal surface 18. Consequently, the ring 20 of the epithelial separator device 12 sits on the eye 10 with its plane also substantially parallel to a limbus of the eye 10. As shown in FIG. 2, the ring 20 defines an internal circumference 22 having a diameter ranging from about 10 to about 12 mm and an external circumference 24 that has a diameter ranging from about 13 to about 16 mm and including a groove 26 (best seen in FIG. 15). The groove 26 is dimensioned wider than the internal diameter 22. Male members 37 formed in the rectangular block A of the separator support 28 snugly fit within and slide within the groove 26 to carry the separator 14 on a determined linear path of travel. A similar groove/male member structure is used in the epithelial separator device 400 of FIGS. 24A-B and 25.

As shown in FIGS. 1A-B, the separator support 28 and the plate 15 are coupled to a movement device, such as an oscillation device 30. The oscillation device 30 generates a force on the plate 15 along a line of force F that is oriented at an angle ranging from 0° to 30° with respect to the top face 31 of the housing 21. The force F is preferably generated via the translational movement of the separator 14 along the direction X caused by the oscillation device 30. The force F is parallel to the direction of the groove so as to have the plate 15 move smoothly in the groove. The force F is also angled in order to control the magnitude of the vertical component of the force F so that cutting is avoided as mentioned previously.

The oscillation device 30 provides translational motion and vibration to the separator 14. In particular, the oscillation device 30 preferably oscillates the plate 15 along direction P shown in FIG. 1A. For such oscillation, the width of the recess can be enlarged by a desired amount and the oscillation device 30 is altered to oscillate the plate 15 along direction P. Note that the oscillation can be along the direction M in the alternative. The frequency of the oscillation along direction P ranges from about 10 Hz to about 10 KHz and the amplitude of such oscillation ranges from 0.8 mm to 2 mm. Such oscillation aids in having the edge 102 separate the layer 16 from the corneal surface 18. In addition, the difference in hardness between the cornea stroma and the epithelium allows the edge 102 to separate the layer 16. Electromagnetic or piezoelectric forces on the plate 15 can provide the oscillation, or external rotating or vibrating wires can provide the oscillation. For example, one end of a shaft can be connected to a motor that rotates the shaft about an axis parallel to the shaft itself. A wire is connected to the other end of the shaft and is rotated by the rotating shaft.

The oscillation device 30 is separately coupled to the separator support 28 so as to generate a constant translational velocity for the support 28 and the distal end 33 and the blunt end 102 of approximately 1.5 mm/s towards the center of the eye 10. In this embodiment, there are two separate motors within the oscillation device 30. One motor closest to the end of the plate 15 is coupled to the plate 15 so as to oscillate the plate 15 along direction M. A second motor is coupled to the previously mentioned motor so that when the second motor translates along direction X, the previously mentioned motor and the coupled plate 15 (and the entire support 14 coupled to plate 15) translate along direction X. Of course, a separate device can be used to translationally move the support 28 instead of the oscillation device 30.

The net effect of the oscillation and translational movement is that the force F is applied by the edge 102 to the layer 16 along direction M and having a magnitude that avoids rupturing the layer 16. The magnitude of such a force can vary from a value of zero, where the plate 15 oscillates along direction P and the separator 14 translates at a constant velocity, to a nonzero value that avoids substantially damaging the epithelial layer 16 and cutting the cornea stroma during the separation process. The latter nonzero force can be generated by oscillating the plate along direction M and/or generating an accelerated translational motion of the separator 14 and plate 15 along direction X.

To maintain the ring 20 on the eye 10, for example during oscillation and translation, the housing 21 and the ring 20 fit snugly on the eye 10 so that a seal is formed and the air within the interior cavity 39 is evacuated via circumferential groove 32 positioned on a side of the eye 10. Suction can be applied to the circumferential groove 32 so that the air within cavity 39 is evacuated to ensure stable mounting of the ring 20 to the eye 10. The lower than atmospheric pressures within evacuated cavity 39 range from 300 mm Hg to 700 mm Hg.

FIGS. 3 and 4 are diagrams showing a side and a top view, respectively, of the eye 10 and the separator 14 located in a second position with respect to the eye. This second position represents the time of initial contact between the eye 10 and both the edge 102 and the end 33. At this position, the plate 15 and edge 102 are initially oscillated along direction P and the separator support 28 is moved along direction X so to have edge 102 puncture through the epithelial layer 16. As described previously, as the separator 14 travels to contact the eye 10, the corneal surface 18 is flattened by the applanator defined by the edge 41 and the bottom surface 35 of the end 33. Simultaneously, the blunt edge 102 begins to push the layer 16. Such pushing causes the layer 16 to be pushed toward the center of the eye. To accommodate the travel of the separator 14, the separator support 28 freely slides in the groove 26, for example, when driven by the oscillation device 30.

FIGS. 5 and 6 are diagrams showing a side and a top view of the eye 10 and the separator 14 located in a third position. As the separator 14 travels along the cornea 10, the epithelial layer 16 is separated from the cornea. The blunt edge 102 of the separator 14 separates the epithelial layer 16 without penetrating or cutting the cornea 18.

More specifically, FIG. 19 is a diagram representing an enlarged side view of the plate 15 and its blunt edge 102 removing the epithelial layer 16 from a Basal membrane 1900 of the eye 10. The epithelial layer 16 is made up of epithelial cells 1902. The epithelial layer 16 overlies the Basal membrane 1900. The Basal membrane 1900 is formed from a lamina densa 1904 of about 50 nm in thickness and an underlying lamina lucida 1906 of about 25 nm in thickness. The lamina densa 1906 overlies a Bowman's layer 1908. The epithelial layer 16 anchors to the Bowman's layer 1908 via a complex mesh of anchoring fibrils (type VII collagen) and anchoring plaques (type VI collagen) that interact with the lamina densa 1904 and the collagen fibrils of the Bowman's layer 1908. The Bowman's layer 1908 overlies a corneal stroma 1910.

The epithelial layer 16 is stratified, possessing 5 to 6 layers of epithelial cells 1902. The epithelial layer 16 is typically about 50 to 60 micrometers in thickness. Adjacent epithelial cells 1902 are held together by desmosomes 1912. The epithelial cells 1902 are held to the underlying Basal membrane 1900 by hemidesmosomes 1914 and anchoring filaments. A bottom surface of the epithelial layer 16 includes numerous microvilli and microplicae, i.e., ridges, whose glycocalyx coat interacts with, and helps to stabilize, a precorneal tear film. New epithelial cells 1902 are derived from mitotic activity in the Basal membrane 1900 layer. New epithelial cells 1902 displace existing cells both superficially and centripetally.

As described above, the plate 15 includes a blunt leading edge 102 to push the epithelial cells 1902 as the plate 15 and blunt edge 102 move under the epithelial layer 16. The plate 15 preferably pushes the epithelial cells 1902 and does not exert a force that could disrupt the intercellular bonds such as the desmosomes 1912. Accordingly, the plate 15 is able to separate the epithelial layer 16 substantially in one piece without cutting the cornea so that it can be transferred back onto its original area of rest upon the tissue that remains after the Basal membrane 1900 once the laser ablation process is finished as will be described below. Note that the point of separating the epithelial layer 16 has been found to often occur at the border between the lamina densa 1904 and the lamina lucida 1906. The plate 15 preferably pushes the bottom two to three layers of epithelial cells 1902 which probably contain a majority of the shear strength of the epithelial layer 16.

FIGS. 7 and 8 are diagrams showing a side and a top view of the eye 10 and the separator 14 located in a fourth position. In one embodiment, the travel of the separator 14 is controlled so that a circular-like area of the separated epithelial layer 16 is formed that is concentric with either the limbus or the pupil (pupil is not generally in the center of the cornea; in many eyes there can be a slight eccentricity). The movement of the separator 14 and its blunt edge 102 is programmed so that the blunt edge 102 stops at a desired position, such as just prior to forming layer 16 in the shape of a circle. Such stoppage creates a hinged area between the portion of the layer 16 that has been separated and the portion of the layer 16 that has not been separated from corneal surface 18. At this stage, the separated portion of the layer 16 has the shape of a “D”, wherein of course the straight portion of the “D” is much smaller than the height of the “D.” For example, during the final phase of the gradual movement shown in FIGS. 1-8, the separator 14 and the blunt edge 102 stop movement at a point slightly before complete separation of a circular disk so that a D-shaped epithelial disk 34 is formed that is attached to a hinge located at an edge 36 of the layer 16 located at a left portion of the eye as shown in FIG. 8. In another embodiment, the epithelial disk 34 is completely detached from the corneal surface 18, for example, as described below.

Note that the above removal process can be done in combination with a handheld spatula that lifts/manipulates the layer 16. The handheld spatula is similar to the one used during the previously described LASEK procedure. In addition, the entire removal process is mechanical in that no chemicals, such as alcohol, are used to loosen the epithelial layer 16.

FIG. 9 is a diagram showing a top view of the eye 10 and the separator 14 located in a retracted position after the epithelial disk 34 as been formed. After the separator 14 is retracted, suction to the circumferential groove 32 is turned off and the epithelial separator device 12 is removed from the eye 10. Referring also to FIG. 10, after the epithelial separator device 12 is removed, a deepithelialized area 38 is exposed that corresponds to a shape and size of the area that the separator 14 contacted during travel. At this point, the surgeon centers the laser ablation applied to the area 38 according to the laser manufacturer.

FIG. 11 shows a top view of the eye 10 after laser ablation is performed on the deepithelialized area 38. The laser ablation forms an irradiated area 40 on the eye 10. Referring to FIG. 12, thereafter, the epithelium disk 34 is replaced on the corneal surface 18 of the eye 10 to aid in the healing process. Note that replacing the epithelium disk 34 can be performed by using a well known spatula that is commonly used to manipulate a LASIK flap. The spatula may also be used to lift and/or manipulate the epithelial layer 16 during the separation process. Referring to FIG. 13, once replaced on the corneal surface 18, the epithelium disk 34 is preferably smoothly stretched into place via such well known surgical instruments as manipulators, spatulas, forceps, or a sponge.

FIG. 21 is a diagram showing a side view of an embodiment of a leading edge 2100 that can be used with a plate 15′. The plate 15′ is similar in structure with the plate 15 of FIGS. 1-9, except that it uses the leading edge 2100. The plate 15′ is supported by separator support 28 so that the plate 15′ and separator support 28 define another embodiment of a separator to replace the separator 14 of FIG. 1. The plate 15′ is moved by oscillation device 30 in the same manner as plate 15 as described previously with respect to FIGS. 1-9. During the removal of the epithelial layer 16, the bottom planar surface 3000 of the plate 15′ is positioned adjacent and parallel to the cornea underlying the Basal membrane 1900 (if it exists) while the flat leading edge 2100 contacts the layer 16. Such an orientation is inherent in order to avoid cutting of the Basal membrane 1900 or cornea stroma. Accordingly, the planar leading edge 2100, the top surface 3002 and the bottom surface 3000 can be treated together as defining a blunt edge with respect to the epithelial layer 16 and the stroma since the epithelial layer 16 is moved without substantial damage and the stroma is not damaged during movement of the bottom surface 3000 and leading edge 2100. The bottom surface 3000 acts as an applanator as the leading edge 2100 moves to remove the layer 16. The top surface 3002 acts as a support surface upon which the separated epithelial layer 16 can lie during the separation process. The leading edge 2100 of the separator 14 should not have a width, w, that is too large such that it will reduce the consistency with which the epithelial layer 16 is penetrated. The leading edge 2100 preferably includes a width w ranging from 5 to 25 micrometers, and more preferably a width w that is about 15 micrometers.

FIG. 22 is a diagram showing a side view of a second embodiment of a leading edge 2100′ that can be used with a plate 15″. The plate 15″ is similar in structure with the plate 15 of FIGS. 1-9, except that it uses the leading edge 2100′. The plate 15″ is supported by separator support 28 so that the plate 15″ and separator support 28 define another embodiment of a separator to replace the separator 14 of FIG. 1. The plate 15″ is moved by oscillation device 30 in the same manner as plate 15 as described previously with respect to FIGS. 1-9. During the removal of the epithelial layer 16, the bottom planar surface 3000′ of the plate 15″ is positioned adjacent and parallel to the cornea underlying the Basal membrane 1900 (if it exists) while the leading edge 2100′ contacts the layer 16. Such an orientation is inherent in order to avoid cutting of the Basal membrane 1900 or cornea stroma. Accordingly, the leading edge 2100′, the top surface 3002′ and the bottom surface 3000′ can be treated together as defining a blunt edge with respect to the epithelial layer 16 and the stroma since the epithelial layer 16 is moved without substantial damage and the stroma is not damaged during movement of the bottom surface 3000′ and leading edge 2100′. The leading edge 2100′ is rounded instead of flat. The leading edge 2100′ has a width w similar to that of the leading edge 2100 of FIG. 21. The leading edge 2100′ has a radius of curvature, r, having a value that ranges from 1 micron to 20 microns. The bottom surface 3000′ acts as an applanator as the leading edge 2100′ moves to remove the layer 16. The top surface 3002′ acts as a support surface upon which the separated epithelial layer 16 can lie during the separation process.

FIG. 23 is a diagram showing a side view of a third embodiment of a leading edge 2100″ that can be used with a plate 15′″. The plate 15′″ is similar in structure with the plate 15 of FIGS. 1-9, except that it uses the leading edge 2100″. The plate 15′″ is supported by separator support 28 so that the plate 15′″ and separator support 28 define another embodiment of a separator to replace the separator 14 of FIG. 1. The plate 15′″ is moved by oscillation device 30 in the same manner as plate 15 as described previously with respect to FIGS. 1-9. During the removal of the epithelial layer 16, the bottom planar surface 3000″ of the plate 15′″ is positioned adjacent and parallel to the cornea underlying the Basal membrane 1900 (if it exists) while the leading edge 2100″ contacts the layer 16. Such an orientation is inherent in order to avoid cutting of the Basal membrane 1900 or cornea stroma. Accordingly, the leading edge 2100″ and the bottom surface 3000″ can be treated together as defining a blunt edge with respect to the epithelial layer 16 and the stroma since the epithelial layer 16 is moved without substantial damage and the stroma is not damaged during movement of the bottom surface 3000″ and leading edge 2100″. The leading edge 2100″ is constructed, for example, by bending the leading edge 2002 of the blade 2000 shown in FIG. 20. The leading edge 2100″ preferably includes a diameter of about 5 to 25 micrometers, or a radius between about 2 to 13 micrometers, and more preferably includes a diameter of 15 micrometers. The bottom surface 3000″ acts as an applanator as the leading edge 2100″ moves to remove the layer 16. The top surface 3002″ acts as a support surface upon which the separated epithelial layer 16 can lie during the separation process.

FIGS. 24A-B and 25 show a second embodiment of an epithelial separator device 4000. The epithelial separator device 4000 includes a housing 4002 made of a rigid material, such as a metal. The housing 4002 defines a track 4004 and a drive coupling 4006 integrally attached to one another. The track 4004 is defined by a planar surface 4008 and two vertical side walls 4010 and 4012 that are spaced from each other by a distance of approximately 14 mm. At one end of the track 4004 a circular opening 4014 having a diameter of approximately 11 mm is formed in the surface 4008. On the bottom of the surface 4008, a 19 mm diameter annular skirt 4011 is formed that is centered about the opening 4014. The skirt 4011 is made of the same material as the surface 4008 and integrally attached thereto. Within the skirt 4011 are seven arc-shaped protrusions 4016 as shown in FIG. 23B that extend from the bottom of the surface 4008. The protrusions 4016 are of equal length, are equi-angularly spaced from one another and are centered about the opening 4014. Each of the protrusions 4016 has a beveled free end that is curved so as to match the external radius of curvature of a portion of eye 10 when contacting the cornea. The protrusions 4016 perform the functions of 1) keeping the conjuctiva in place and 2) uniformly distributing the vacuum in the circumference of the skirt 4011. The conjuctiva is tissue like a membrane that covers the eye at the portion where the skirt 4011 attaches to. The conjuctiva sometimes may be somehow loose and therefore rise when vacuum is applied within skirt 4011. If it rises in the suction ring it can go to the suction port (where tube 4034 goes into the suction ring) and block it. In that case the vacuum would be not distributed evenly around the suction ring and the whole device would not be stable on the eye.

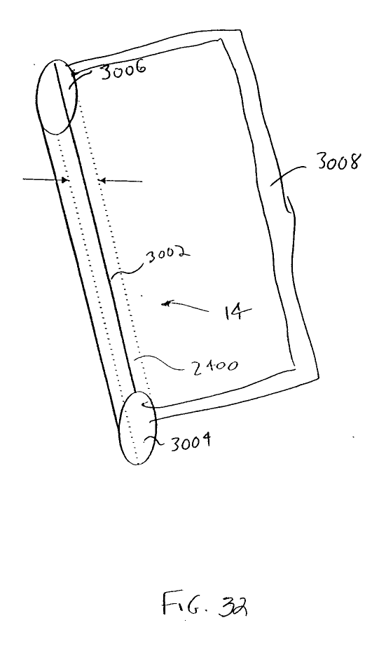

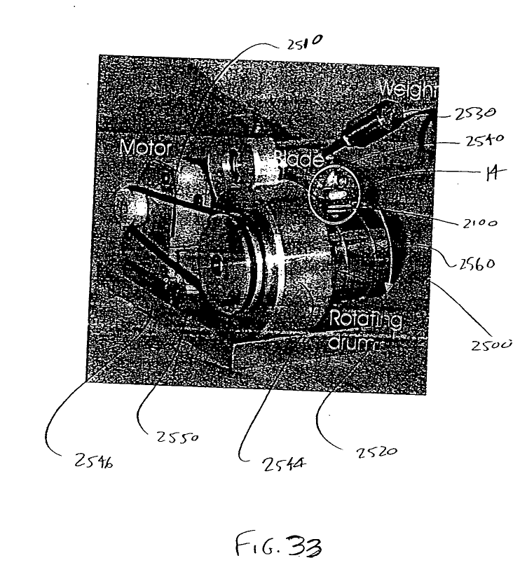

The device 4000 includes a separator 4018 as shown in FIGS. 24-25. Other separators can be used in device 4000 instead of separator 4018. For example, the separator 14 of FIGS. 1-9, the separator support 28 containing the blades 15′, 15″ and 15′″ of FIGS. 21-23 and the separators 14′ and 14″ of FIGS. 30-31 can be used in device 4000. As shown in FIG. 25, the separator 4018 includes a blunt leading edge 2100′″ formed at the end of planar surfaces 2200 and 2210. The planar surfaces 2200 and 2202 are angularly separated from one another by an amount ranging from 20 to 40 degrees, preferably approximately 26 degrees. The bottom planar surface 2202 is oriented in a range from 10 to 20 degrees, preferably approximately 14 degrees, with respect to the planar bottom surface 2210 (width along direction of movement approximately 300 μm) and has a length, d, of approximately 1.5 mm. The leading edge 2100′″ pushes the layer 16 while the bottom surface 2210 acts as an applanator and flattens the eye while the separator 14 moves. Since the leading edge 2100′″ and surfaces 2200, 2202 and 2210 do not substantially damage the epithelial layer 16 and do not damage or cut the stroma during the separation process, the leading edge 2100′″, surface 2210 and at least portions of surfaces 2200 and 2202 can be viewed jointly as a blunt edge. In addition, the surface 2200 can act as a support surface for supporting the separated epithelial layer during the separation process.

The separator 4018 is positioned within a metallic holder 4020 that includes: 1) a planar support surface 4022 that slides upon surface 4008, 2) a U-shaped side wall 4024 and 3) a pivotable top surface 4026. The support surface 4022 includes parallel bottom side male members, similar to male member 37 of FIG. 15, that engage corresponding bottom grooves formed in the side walls 4010, 4012, similar to groove 26 of FIG. 15, After the top surface 4026 is pivoted to an open position, the separator 4018 is slid into the cavity defined by the holder 4020 so that the rectangular recess 4027 is snugly engaged by rectangular male member (not shown) formed on the top portion of surface 4022 and a leading edge of the separator 4018 extends out of the holder 4020. Once properly positioned, the top surface 4026 is pivoted to a closed position wherein the top surface 4026 lies just above the top surface of the separator 4018. Next, the separator 4018 is held in place upon tightening a screw 4028 of the top surface 4026 that threadingly engages an opening formed in the body of the holder 4020 located below the closed top surface 4026. Such a screwing action causes the bottom surface of the surface 4026 to engage with the top surface of the separator 4018 and so trapping the separator 4018 within the holder 4020.

The separator 4018 is designed to contact and remove an epithelial layer 16 without making an incision in corneal tissue located below the epithelium. The separator 4018 removes an epithelial layer 16 located above a corneal surface 18 of the eye 10 in a manner similar to that described previously with respect to separator 14 with respect to FIGS. 1-9. The separator 4018 is not sharp enough to excise corneal tissue during operation of the epithelial separator device 4000.

As shown in FIG. 24A, the separator 4018 is coupled to an oscillation device 4030 via a rod 4032 that has one of its ends attached to the rear of the holder 4020. The other end of the rod 4032 passes through an opening formed in the drive coupling 4006 and is directly coupled to the oscillation device 4030. The other end of the rod 4032 defines a stop that limits the distance that the rod 4032 can be translated relative to the coupling 4006. When the stop engages the portions of the drive coupling 4006 that define the opening, the leading edge of the separator 4018 just passes past the opening nearest the end 4034 of the device 4000. The oscillation device 4030 generates a force on the separator 4018 parallel to the surface 4008.

In operation, the skirt 4011 and the protrusions 4016 are placed on the exterior surface of the eye 10 so as to form a seal and so that the surface 4008 is parallel to a limbus of the eye 10. To maintain the skirt 4011 on the eye 10, for example during oscillation, air within the interior cavity is evacuated via a portal 4034 positioned on a side of the eye 10. Suction can be applied via portal 4034 so that the air within cavity 39 is evacuated to ensure stable mounting of the skirt 4011 to the eye 10.

Note that it is known that applying suction can result in tissue, such as conjunctiva tissue, being displaced from the eye so as to plug up the portal 4034. To overcome this, a C-shaped guard 5000 is used as shown in FIG. 26. The guard 5000 is made of a resilient material, such as a sterilizable metal or hard plastic, and has a diameter that is slightly larger than that of the interior of the skirt 4011. Thus, when the guard 5000 is placed within the cavity defined by skirt 4011, the bottom 5002 of the guard 5000 engages the bottom portion of skirt 4011 in a spring-like manner so as to create a seal. The top portion 5004 of the guard is annular like in shape and is slightly recessed inward with respect to the exterior of the bottom 5002. The top portion 5004 has several rectangular openings 5006 formed which define a plurality of arc-like guards 5008. When the guard 5000 is inserted within the skirt 4010, one of the guards 5008 is positioned in front of the portal 4034. In operation, the guard 5000 allows suction to be performed via the passages defined by the recessed top portion 5004, the skirt 4011 and the openings 5006. The guards 5008 intercept/block tissue before it can clog up the portal 4034. Note that the guard 5000 can be used with the device of FIG. 1 in a similar manner.

Once the device 4000 is positioned upon the eye, the oscillation device 4030 is operated in a manner similar to that described previously with respect to the oscillation device 30 so as to provide translational motion and vibration to the separator 4018. Furthermore, the epithelial layer 16 is removed in a manner similar to that described previously with respect to the device of FIGS. 3-8 without cutting the cornea 18. Note that while ablation of the cornea 10 is performed by the separator 4018 alone, it is possible to perform ablation in a serial manner with the separator 4018 by employing a metal applanator 6000 that is attached to and spaced in front of the holder 4020 as shown in FIGS. 27-29. The applanator 6000 has a width of about 2 mm as measured along the direction X. As shown in FIG. 29, the lower portion of the applanator 6000 is rounded and polished. As shown in FIGS. 27 and 28, the applanator 6000 is in the shape of an “H” with the vertical parts 6002 being bent so that ends of the parts 6002 fit in and slide within the previously mentioned bottom grooves of the side walls 4010, 4012. Note that the male members 6004 of the support surface 4022 extend towards the parts 6002 so that when metal holder 4020 moves forward, the male members engage the vertical parts 6002 and push the applanator 6000 as well. However, when the support surface 4022 is retracted in the opposite direction, the applanator 6000 does not move and so is not retracted.

In operation, the cornea is first applanated by applanator 6000 and then applanated again by the separator 4018 while the layer 16 is being removed in the manner described previously. The applanator flattens the eye before the blunt edge reaches the eye Note that applanator 6000 can be adapted to be used with the device shown in FIG. 1 so as to operate in a similar manner as described above.

After the layer 16 is removed, the separator 4018 is retracted in a manner similar to that shown in FIG. 9. After the separator 4018 is retracted, suction is turned off and the epithelial separator device 4000 is removed from the eye 10. After the epithelial separator device 4000 is removed, a deepithelialized area 38, such as shown in FIG. 10, is exposed that corresponds to a shape and size of the area that the separator 4018 contacted during travel.

Next, laser ablation of the exposed area is performed so as to form an irradiated area 40 on the eye 10 as shown in FIG. 11. Referring to FIG. 12, thereafter, the epithelium disk 34 is replaced on the corneal surface 18 of the eye 10 to aid in the healing process. Referring to FIG. 13, once replaced on the corneal surface 18, the epithelium disk 34 is preferably smoothly stretched into place. Note that the disk 34 can be removed using the devices described previously with respect to FIGS. 14-18.

FIG. 30 shows a side view of a second embodiment of a separator 4018′ that can take the place of the separator 4018 (see FIG. 25) in the epithelial separator device 4000 of FIGS. 24A-B. In particular, the separator 4018′ can be slid into the cavity defined by the holder 4020 so that the rectangular recess 4027 is snugly engaged by rectangular male member (not shown) formed on the top portion of surface 4022 and a leading edge 2100′ of the separator 4018′ extends outward. It is also envisioned that the separator 4018′ can take the place of the separator 14 and the end 33 of the epithelial device 12 of FIGS. 1-9. The blunt leading edge 2100′ of separator 4018′ is rounded and formed at the ends of planar surfaces 2200′ and 2202′ that are angularly separated from one another by an amount ranging from 10 to 30 degrees, preferably approximately 25 degrees. The bottom surface 2202′ is oriented approximately 20 to 30 degrees, preferably 26 degrees, with respect to the top surface 31 of the housing 21 and has a length, d, of approximately 1.5 mm. The leading edge 2100′ pushes the layer 16 while the bottom surface 2202′ acts as an applanator and flattens the eye while the separator 14 moves. Since the leading edge 2100′ and surfaces 2200′ and 2202′ do not substantially damage the epithelial layer 16 and do not damage or cut the stroma during the separation process, the leading edge 2100′″ and at least portions of surfaces 2200′ and 2202′ can be viewed jointly as a blunt edge. In addition, the surface 2200′ can act as a support surface for supporting the separated epithelial layer during the separation process.

FIGS. 31A-B show side views of a third embodiment of a separator 4018″ that can take the place of the separator 4018 (see FIG. 25) in the epithelial separator device 4000 of FIGS. 24A-B. In particular, the separator 4018″ is slid into the cavity defined by the holder 4020 so that the rectangular recess 4027 is snugly engaged by rectangular male member (not shown) formed on the top portion of surface 4022 and a leading edge 2100″ of the separator 4018 extends outward. It is also envisioned that the separator 4018″ can take the place of the separator 14 of the epithelial device 14 of FIGS. 1-9. The embodiment is disclosed in a U.S. Provisional Patent Application filed on Aug. 6, 2004 and having the title “Separator for Corneal Epithelium” listing Scott M. Hampton, Stephen Woods and Harilaos Ginis as inventors and having an attorney docket no. of 2N04.1-040, the entire contents of which are incorporated herein by reference. The blunt leading edge 2100″ of separator 4018″ is formed at the end of planar surfaces 2204 and 2206 that are angularly separated from one another by an amount ranging from 30 to 60 degrees, preferably approximately 40 degrees. The bottom surface 2206 has a length, b, of approximately 0.3 mm and is oriented parallel to surface 31. The bottom surface 2206 is oriented 20 to 40 degrees, preferably approximately 26 degrees, with respect to an angled bottom surface 2208, which has a length, c, of approximately 1.2 mm. The leading edge 2100″ pushes the layer 16 while the bottom surface 2206 acts as an applanator and flattens the eye while the separator 14 moves. Note that bottom surface 2206 enhances applanation when compared with when the blunt edges of separators of FIGS. 25 and 30 are used for applanation. Since the leading edge 2100″ and surfaces 2204, 2206 and 2208 do not substantially damage the epithelial layer 16 and do not damage or cut the stroma during the separation process, the leading edge 2100″, surface 2206 and at least portions of surfaces 2204 and 2208 can be viewed jointly as a blunt edge. In addition, the surface 2204 can act as a support surface for supporting the separated epithelial layer during the separation process.

A variation of the separator 4018′″ is shown in FIG. 31C, wherein the distal end of the separator 4018′″ that includes surfaces 2204, 2206 and 2208 is replaced by a separator 4018“ ” that is similar to separator 4018′″ except that its distal end material has a trapezoidal-like side cross-sectional shape. The material includes surfaces 2204′, 2206′ and 2208′ that define leading edge 2100′″. Surface 2206′ has a length b′ of about 300 μm. Since the leading edge 2100′″ and surfaces 2204′, 2206′ and 2208′ do not substantially damage the epithelial layer 16 and do not damage or cut the stroma during the separation process, the leading edge 2100′″, surface 2206′ and at least a portion of surface 2204′ can be viewed jointly as a blunt edge. In addition, the surface 2204′ can act as a support surface for supporting the separated epithelial layer during the separation process.

Note that the leading edges 2100, 2100′, 2100″ and 2100′″ of FIGS. 25 and 30-31 are formed from separators 4018, 4018′, 4018″ and 4018′″, respectively, that are made of a number of rigid and sterilizable materials, such as metals and plastics. The lines of intersection between two connected surfaces can be dulled by placing the separators in a container containing glass beads, wherein the container is rotated so that the tumbling of the glass beads lessens the sharpness of the lines of intersection. The separators 4018, 4018′, 4018″ and 4018′″ with their respective leading edges 2100, 2100′, 2100″, 2100′″ are able to separate the epithelial layer 16 substantially in one piece without cutting the cornea so that it can be transferred back onto its original area of rest upon the tissue that remains after the laser ablation process is finished as will be described below. Note that Basal membrane 1900 may be removed once the laser ablation process is finished as will be described below.

FIG. 32 is a diagram showing a perspective view of a wire 2400 that could be used as a replacement for the separators 14, 4018, 4018′, 4018″ and 4018′″ according to a preferred embodiment. The wire 2400 includes a generally elliptical or circular cross-sectional shape. The wire 2400 includes a leading edge 3002 with a width of about 5 to 25 micrometers. The wire 2400 is preferably manufactured from a material that is strong enough to push the epithelium without breaking. Exemplary wire materials include titanium and its alloys, tungsten and its alloys, steel alloys and carbon fibers. The two ends 3004 and 3006 of the wire 2400 are preferably attached to a yoke 3008 that is coupled to the oscillation device 14. The yoke 3008 maintains tension in the wire 2400 so that the leading edge 3002 remains relatively straight while it is pushing the epithelial layer 16.

Note that in all of the embodiments of the separators shown in FIGS. 1-32, the separators and associated oscillation devices are positioned so that they move the separators towards the bridge B of the nose of the patient (see FIG. 2). This movement causes the epithelial flap to be positioned on the bridge of the nose. Such a position can lead to damage to the flap should the patient blink his or her eyes.

As an alternative, the separators and oscillation devices can be rotated by 90 degrees so that the separators move towards the eyebrow of the patient. In this case, the epithelial flap would be positioned in the more advantageous position on the eyebrow E of the patient (see FIG. 2). The oscillation devices of FIGS. 1-32 may contact the cheekbone of the patient which could hinder positioning the ring on the eye. This can be corrected by either making the suction ring deeper or redesigning the oscillation device housing structure so that it avoids the cheek.

Note that in all of the embodiments of the separators shown in FIGS. 1-32, the oscillation devices can be altered to be controlled by a closed loop control system 7000. Such a control system would be designed so as to control the distance traveled by the separator along the direction X. In addition, the control system 7000 would control the velocity of the separator along direction X so that the velocity is constant during the entire separation process, even when the separator contacts the stroma and epithelial layer. The control system 7000 would also control the frequency of oscillations along direction M or P so that the frequency is constant during the entire separation process, even when the separator contacts the stroma and epithelial layer.

FIG. 14 is a diagram showing a side view of the eye 10 and an embodiment of an epithelial separator device 12′ that includes a rotating drum 42. The device 12′ essentially combines the structure of the epithelial separator device 12 of FIGS. 1-9 with a drum structure that will be discussed below. In particular, the device 12′ includes a U-shaped yoke 51 that supports the drum 42 therebetween via an axle 53. The axle 53 can be supported by both legs of the yoke 51, like a bicycle wheel, or by just one of the legs, like a paint roller. To rotate the drum 42, the epithelial separator device 12′ may include a rotating gear 44. The gear 44 could also be used to provide movement to the separator support 28′ that is similar to the separator support 28 shown in FIG. 1B. The separator 28′ is similar in structure to separator 28 of FIG. 1, except that it includes the yoke 51.

Referring also to FIGS. 15 and 16, front and top views, respectively, of the epithelial separator device 12′, the rotating gears 44 could be symmetrically placed on both sides of the separator support 28′ of the separator 14′. The oscillating device 30 can provide for rotation of the gears 44 and the gears 44 can travel on rails, for example toothed rails, which run parallel to the groove 26. In addition, the drum 42 acts as an applanator as shown in FIG. 14. A second applanator, similar to applanator 6000 of FIGS. 27-29 can be used in device 12′ so as to be positioned prior and in series with the drum 42.

Since a typical thickness of an epithelial disk 36 includes about 50 microns, to preserve an epithelial disk 36, a separated epithelial disk 36 is rolled onto the drum 42. The drum 42 can include a diameter ranging from about 3 to about 9 mm and a length of about 12 mm. Referring also to FIG. 17, in one embodiment, to maintain integrity of the epithelial disk 36, the drum 42 can be coated with a hydrating and/or a conditioning substrate. While the layer 16 could be adhered to drum 42 without the use of a substrate, the substrate does provide controllable adhesion of layer 16 to the drum 42 The hydrating and/or conditioning substrate can include, for example, HEMA contact lenses, tissue culture media, silicone and biocompatible hydrogels. The hydrating and/or conditioning substrate can be removed from the drum after the epithelial disk 36 attaches on to the drum. Thereafter, the epithelial disk 36 can be removed from the drum 46 and replaced on the corneal surface 16, as described above.

FIG. 18 shows another embodiment of a drum 42′ that can replace the drum 42 of the device 12′ of FIGS. 14-17. The drum 42′ includes apertures 46 and a connector 48 that connects to a suction source (not shown). By applying suction to the apertures 46 of the drum 42, the epithelial disk 36 can be rolled onto the drum 42. Thereafter, the epithelial disk 36 can be removed from the drum 46 and replaced on the corneal surface 16, as described above.

FIG. 35 shows a side view of one embodiment of a device 2700 for separating and preserving the epithelial layer 16 that has been pushed/separated by the separator devices 12 and 4000 of FIGS. 1-9 and 24-25 using a blunt edge such as shown in FIGS. 21-23 and 30-31. The device 2700 includes a body 2705, a first drum 2720 and a second drum 2730, and a belt 2730 connecting the first drum 2720 to the second drum 2730. The device 2700 accommodates a substrate, such as film 2740. Film 2740 is used to substantially preserve the epithelial layer 16 when the epithelial layer 16 is removed from the eye 10. The film 2740 can be held to the drum 2710 with a bar or clip 2750. Alternatively, the film 2740 can serve to connect the drums 2720 and 2730 and therefore eliminate the use of belt 2730.

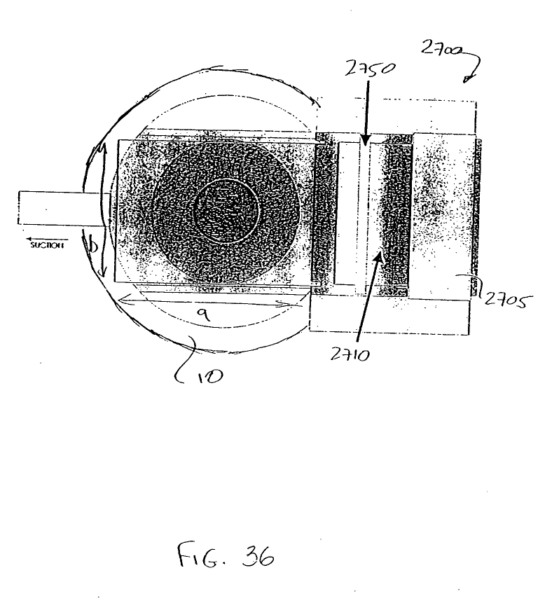

FIG. 36 shows a top view of the device 2710 and how the device 2700 is used with the clip 2750. In one embodiment, the film 2740 is rolled onto the drum 2710 and under the clip 2750 (see also FIG. 35). The first drum 2710 turns as the second drum 2720 turns since they are connected by the belt 2730. The film 2740 lays on the belt 2730 and moves as the first drum 2710 and the second drum 2720 move. The film 2740 preferably removably adheres to the belt 2730 through cohesion. The use of two drums 2710 and 2720 allows the smaller drum 2720 to go very near the separators of FIGS. 1-9, 21-23 and 30-31 and so can act as an applanator. In addition, the belt 2730 is of sufficient size to accommodate the entire removed epithelium layer 16.

The film 2740 includes an outer surface 2760. The outer surface 2760 is constructed to adhere to the epithelial layer 16 to provide mechanical stability to the epithelial layer 16 when the epithelial layer 16 is separated from the eye 10. The film 2740 includes a natural or synthetic polymer. An exemplary polymer includes HEMA (poly-2hydroxy-ethyl-methacrylate). The film 2740 includes a thickness from about 20 to about 100 micrometers. If the film 2740 is in the shape of a strip of film, a length (a) and a width (b) of the film 2740 is preferably longer and wider than the diameter of a separated epithelial layer 16.

The film 2740 is preferably hydrated to adhere the epithelial layer 16 to the film 2740. The level of hydration of the film 2740 controls adhesion to the film 2740. The hydrated film 2740 also helps to keep cracks from forming in the removed epithelial layer 16, and to help avoid the removed epithelial layer 16 from being torn or shrinking. In one embodiment, a surface of the epithelial layer 16 is dried, for example, with a sponge or with a compressed air flow. Thereafter, the film 2740 is placed on the epithelial layer 16. The epithelial layer 16 adheres to the dry film 2740 because of the difference in hydration levels between the epithelial layer and the film. Thereafter, the separator 14 is used to separate the epithelial layer 16 from the corneal surface 18. The film 2740 and its attached epithelial layer 16 are rolled onto the first and second drums 2710, 2720.

It should be appreciated that the strip of film 2740 does not have to be applied with the device 2700 and that the strip does not need to include a coating. Moreover, the film 2740 can be applied before or after removal of the epithelial layer 16, and can be manually applied instead of using the device 2700.

The film 2740 can include other shapes such as the shape of a disc. A way to attach the epithelial layer 16 to a disc, such as a contact lens, is to separate the epithelial layer 16 from the eye 10 and remove the epithelial layer 16 to the side. The epithelial layer 16 is then smoothed with a sponge and dried with the sponge, compressed air or both. Thereafter, the removed epithelial layer 16 is placed on the film 2740. The epithelial layer 16 and the film 2740 are then dried, for example, with compressed air. After about 30 seconds of drying, the epithelial layer 16 is adhered to the film 2740 and can be more easily manipulated with a reduced risk of damage.

After the epithelial layer 16 is adhered to film 2740, the laser is applied to the surface of the cornea in a manner similar to that described previously with respect to PRK. Once the laser treatment has been completed, the corneal surface is dried and the film 2740 is laid upon the eye 10 so that the epithelial layer is laid back substantially into its original place upon the eye 10. Next, drops of water are applied to the anterior surface of the film 2740. The applied water diffuses in the film resulting in the film and the side of the film adjacent to the epithelial layer 16 being wetted. At this stage, the film 2740 is lifted off of the epithelial layer 16 and the epithelial layer 16 is attached to the eye 10.

FIG. 33 shows a perspective view of an embodiment of an exemplary machine 2500 that is used to condition a separator 14, 4018, 4018′, 4018″. The machine 2500 conditions the separator 14, 4018, 4018′, 4018″ by changing a sharp edged separator to include a generally bent edge, for example, like the front edges of the separators 14, 4018, 4018′, 4018″ shown in FIGS. 23, 25 and 30-31.

FIG. 34A shows a front view and FIG. 34B schematically shows a side view of the machine 2500 and separator 14, 4018. Referring to FIGS. 33 and 34, the machine 2500 includes a motor 2510, a rotating cylinder 2520, a weight 2530, or other way to hold the blade/separators 14, 4018 associated with leading edges 2100 and 2100′ of FIGS. 21 and 22, and a blade/separator holder 2540. The motor 2510 and a housing 2544 of the cylinder 2520 rest on a platform 2546. The separators associated with leading edges 2100 or 2100′ of FIGS. 21-22, respectively, are held by, for example, a clamp. In each case, the leading edge 2100, 2100′ is substantially parallel to the axis of rotation of cylinder 2520. As shown in FIG. 34B, the blade's plane, B, forms an angle ψ between 0 and 20 degrees with the plane, P, defined by the axis of the cylinder 2520 and the blade's edge. The motor 2510 connects to the cylinder 2520 via a belt 2550 to rotate the cylinder 2520. In another embodiment, the motor 2510 connects directly to the cylinder 2520 to rotate the cylinder.

The cylinder 2520 includes a helical wire 2560. The helical wire 2560 and the cylinder 2520 are manufactured from steel. This helical wire 2560 serves as a helical protrusion of the rotating drum. This helical protrusion has a pitch equal to the length of the leading edge of the separator/blade. The helix causes only one point of the leading edge of the separator/blade to be conditioned at any given moment (the point of contact between the leading edge and the helical wire). As the helical wire 2560 rotates along with drum 2520, the point of contact travels along the length of the leading edge, but the amount of conditioning is equal across the entire length of the leading edge. The amount of weight 2530, and the running time and rotations of the cylinder 2520 vary the shape and width of the leading edge 2100 of the associated separator. For example, increasing the weight 2530 will result in more bending. In one embodiment, a preferred separator has been conditioned by asserting 20 mN of force on the separator to the cylinder 2520 and operating the cylinder for about 45 second at 0.7 (seven-tenths) rotations/second. The leading edge formed corresponds to leading edge 2100″ shown in FIG. 31A.

While the invention has been described above by reference to various embodiments, it will be understood that many changes and modifications can be made without departing from the scope of the invention. It is therefore intended that the foregoing detailed description be understood as an illustration of the presently preferred embodiments of the invention, and not as a definition of the invention. It is only the following claims, including all equivalents, which are intended to define the scope of this invention.

1. A separator comprising:
a body; and
a leading edge portion comprising:
an upper planar surface oriented at an angle ranging from approximately 40° to approximately 90° relative to a horizontal plane; and
a lower planar surface oriented at an angle ranging from
approximately 0° to approximately 30° relative to said horizontal plane, wherein said upper planar surface and said lower planar surface define a blunt leading edge therebetween and said leading edge portion is made of a material that has a hardness so as to separate an epithelial layer of an eye from an cornea stroma of said eye without substantially damaging said epithelial layer and wherein said blunt leading edge is incapable of cutting said stroma.

2. The separator of claim 1, further comprising a third planar surface attached to said lower planar surface.

3. The separator of claim 2, wherein said third planar surface is oriented at an angle ranging from approximately 10 degrees to approximately 20 degrees.

4. A method of processing an eye for a corrective procedure comprising:
moving a separator relative to a cornea, wherein said separator comprises:
a body; and
a leading edge portion comprising:
an upper planar surface oriented at an angle ranging from approximately 40° to approximately 90° relative to a horizontal plane; and
a lower planar surface oriented at an angle ranging from approximately 0° to approximately 30° relative to said horizontal plane, wherein said upper planar surface and said lower planar surface define a blunt leading edge therebetween and said leading edge portion is made of a material that has a hardness so as to separate an epithelial layer of an eye from an cornea stroma of said eye without substantially damaging said epithelial layer and wherein said blunt leading edge is incapable of cutting said stroma.

5. The method of claim 4, wherein during said moving, said blunt leading edge removing substantially all of an epithelial layer in one piece from a cornea and said applanation surface simultaneously ablating said cornea.

6. The method of claim 5, further comprising replacing said removed epithelial layer upon said cornea.

7. A method of processing an eye of a patient for a corrective procedure comprising:
moving a separator relative to a cornea of an eye;
separating an epithelial layer associated with said cornea, wherein said separated epithelial layer defines a hinge on said cornea so that a free end of said epithelial layer is pivoted about said hinge so as to be positioned near an eyebrow of said patient.

8. A method of processing an eye of a patient for a corrective procedure comprising:
moving a separator along a linear direction relative to a cornea of an eye; and
automatically and electronically controlling the distance traveled by said separator along said linear direction.

9. The method of claim 8, further comprising automatically and electronically controlling a velocity of said separator along said linear direction so as to be constant at all times during said method.

10. The method of claim 8, wherein said separator oscillates along a second linear direction, said method further comprising automatically and electronically controlling a frequency of said oscillation along said second linear direction so as to be constant at all time during said method.

11. The method of claim 9, wherein said separator oscillates along a second linear direction, said method further comprising automatically and electronically controlling a frequency of said oscillation along said second linear direction so as to be constant at all time during said method.
